(19) United States
(12) Patent Application Publication
(10) Pub. No.: US 2026/0248933 A1
(43) Pub. Date: Aug. 27, 2026

(54) METHODS FOR TREATING INFLUENZA INFECTIONS

(71) Applicant: Cidara Therapeutics, Inc., San Diego, CA (US)

(72) Inventors: Ozlem K. EQUILS, Santa Rosa, CA (US); Shawn D. FLANAGAN, Escondido, CA (US); Voon ONG, San Diego, CA (US); Taylor SANDISON, Encinitas, CA (US)

(21) Appl. No.: 19/160,564

(22) PCT Filed: Mar. 1, 2024
(86) PCT No.: PCT/US2024/018179

§ 371 Date: Aug. 28, 2025

Related U.S. Application Data
(60) Provisional application No. 63/487,832, filed on Mar. 1, 2023.
(60) Provisional application No. 63/463,664, filed on May 3, 2023.
(60) Provisional application No. 63/539,451, filed on Sep. 20, 2023.
(60) Provisional application No. 63/603,957, filed on Nov. 29, 2023.

(51) Int. Cl. A61K 47/68; A61K 31/351; A61P 31/16

(52) U.S. Cl. CPC A61K 47/6801; A61K 31/351; A61P 31/16

(57) ABSTRACT

Provided herein are methods for inhibiting, reducing, or shortening influenza viral replication or infection in a human subject as well as methods for treating influenza infections or related conditions thereto in a human subject in need thereof. The methods include the administration of a zanamivir-Fc conjugate having the structure of Formula (I) in a dosage of 10 to 900 mg.

### BACKGROUND

The need for novel antiviral treatments for influenza is significant and especially critical in the medical field. Influenza virus, the causative agent of influenza, or the flu, is responsible for three to five million cases of severe illness annually, and approximately 500,000 deaths worldwide. While most people recover completely from influenza in about one to two weeks, others develop life-threatening complications, such as pneumonia. Thus, influenza can be deadly, especially for the young, old, or chronically ill. People with weak or compromised immune systems, such as people with advanced HIV infection or transplant patients, whose immune systems are medically suppressed to prevent transplant organ rejection, are at greater risk for complications relating to influenza. Pregnant women and young children are also at a high risk for complications.

The development of antiviral treatments for influenza has been a continuing challenge. Several influenza antiviral agents have been approved for use in the clinic, and these agents play important roles in modulating disease severity and controlling pandemics while vaccines are prepared. However, drug-resistant strains have emerged to the most commonly used inhibitors.

Influenza antiviral agents largely target proteins presented on the surface of the influenza virus particle. The envelope of the influenza virus contains two immunodominant glycoproteins, hemagglutinin and neuraminidase, that play key roles in viral infection and spread. Hemagglutinin effects attachment of the virus to the host cell through its interaction with surface sialic acids, thereby initiating entry. Neuraminidase is an exo-glycosidase enzyme that cleaves sialic acids (terminal neuraminic acid residues) from glycan structures on the surface of infected host cells, releasing progeny viruses and allowing the spread of the virus from the host cell to uninfected surrounding cells. Inhibition of neuraminidase therefore serves as a pharmacological target for antiviral drugs. Viral neuraminidase inhibitors used to reduce viral spread have been identified, including oseltamivir (Tamiflu™), zanamivir (Relenza™), and peramivir (Rapivab™).

Influenza in transplant recipients remains characterized by prolonged viral shedding, increasing the likelihood of developing drug resistant strains. New, more effective therapies for treating influenza are needed.

### SUMMARY OF THE INVENTION

In a first aspect, the disclosure provides a method for inhibiting, reducing, or shortening influenza viral replication or infection in a human subject. This method includes or consists of subcutaneously or intramuscularly administering a dose of a conjugate of Formula (I) to the human subject in an amount of 10 mg to 900 mg (e.g., 10 mg, 15 mg, 20 mg, 25 mg, 30 mg, 35 mg, 40 mg, 45 mg, 50 mg, 150 mg, 300 mg, 450 mg, or 900 mg), wherein the conjugate of Formula (I) has the following structure:

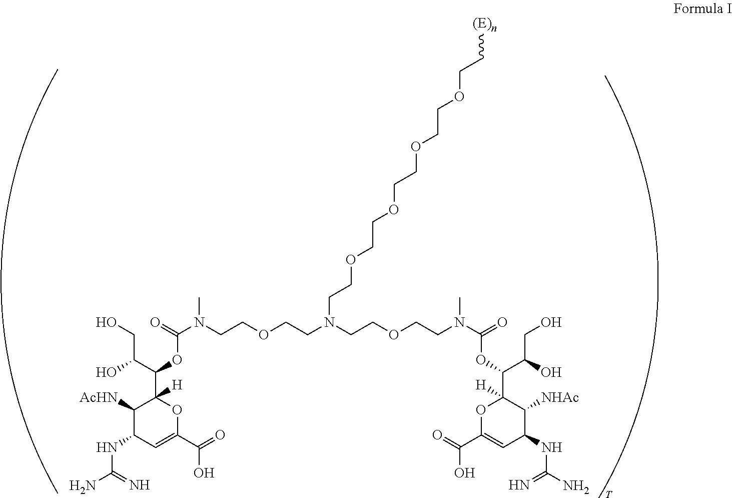

wherein each E is an Fc domain monomer;
n is 2;
T is an integer from 3 to 6; and
the squiggly line indicates a covalent attachment to a nitrogen atom of a solvent-exposed lysine or to a sulfur atom of a solvent-exposed cysteine of an E.

In another aspect, the disclosure provides a method for treating a human subject having or at risk for an influenza infection. This method includes or consists of subcutaneously or intramuscularly administering a dose of a conjugate of Formula (I) to the human subject in an amount of 10 mg to 900 mg.

In another aspect, the disclosure provides methods for reducing the area under the influenza viral load time curve (VL-AUC), for reducing the peak influenza viral load time, reducing the time to a confirmed negative influenza test, reducing total clinical symptoms score (TSS-AUC), reducing peak of TSS, or reducing time to symptom resolution in a human subject (e.g., a human subject having an influenza infection). These methods include or consist of subcutaneously or intramuscularly administering a conjugate of Formula (I) to the human subject in an amount of 10 mg to 900 mg.

In another aspect, the disclosure provides a method for inhibiting, reducing, or shortening influenza viral replication or infection in a human subject, wherein the method includes or consists of maintaining a minimum plasma concentration of a conjugate of Formula (I) of at least 300 ng/ml over four to six months. In some embodiments, the method includes or consists of maintaining a minimum plasma concentration of the conjugate of at least 1 μg/mL. In some embodiments, a minimum plasma concentration of at least 300 ng/ml of the conjugate is maintained over six months in the subject. In some embodiments, a minimum plasma concentration of at least 300 ng/ml of the conjugate is maintained over four months in the subject. In some embodiments, a minimum plasma concentration of at least 1 μg/mL of the conjugate is maintained over six months in the subject. In some embodiments, a minimum plasma concentration of at least 1 μg/mL of the conjugate is maintained over four months in the subject.

In some embodiments of any of the above methods, the method consists of a single administration of the conjugate to a human subject. In some embodiments, the human subject is administered a single dose of 10 mg, 15 mg, 20 mg, 25 mg, 30 mg, 35 mg, 40 mg, 45 mg, 50 mg, 150 mg, 300 mg, 450 mg, or 900 mg of the conjugate.

In some embodiments of any of the above methods, the methods include or consist of two or more (e.g., three or four) administrations of the conjugate to a human subject. In some embodiments, the methods include or consist of two administrations of the conjugate to a human subject, wherein the two administrations are from 60 to 120 days apart (e.g., from 75 to 105 days apart).

In some embodiments, a dose for administration includes 10 mg, 15 mg, 20 mg, 25 mg, 30 mg, 35 mg, 40 mg, 45 mg, 50 mg, 150 mg, 300 mg, 450 mg, or 900 mg of the conjugate.

In some embodiments of any of the above methods, the conjugate is Conjugate A, which has the structure provided herein.

In another aspect, the disclosure provides a pharmaceutical composition in unit dosage form, wherein the pharmaceutical composition includes a conjugate of Formula (I) (e.g., Conjugate A) in an amount of 10 mg to 900 mg. In some embodiments, the pharmaceutical composition contains 10 mg, 15 mg, 20 mg, 25 mg, 30 mg, 35 mg, 40 mg, 45 mg, 50 mg, 150 mg, 300 mg, 450 mg, or 900 mg of the conjugate.

In another aspect, the disclosure provides a kit that includes the pharmaceutical composition described above and instructions for use in a method of any of the methods described herein.

In some embodiments of any of the above aspects, the conjugate includes an Fc domain that includes a protein having an amino acid sequence at least 95% identical to the sequence of any one of SEQ ID NO: 62, SEQ ID NO: 63, SEQ ID NO: 64, SEQ ID NO: 67, SEQ ID NO: 68, SEQ ID NO: 72, SEQ ID NO: 73, SEQ ID NO: 76, SEQ ID NO: 77, SEQ ID NO: 80, SEQ ID NO: 81, SEQ ID NO: 82, SEQ ID NO: 85, SEQ ID NO: 86, SEQ ID NO: 90, SEQ ID NO: 91, SEQ ID NO: 94, or SEQ ID NO: 95. In some embodiments, the conjugate includes an Fc domain that includes a protein having an amino acid sequence of any one of SEQ ID NO: 63, SEQ ID NO: 64, SEQ ID NO: 67, SEQ ID NO: 68, SEQ ID NO: 72, SEQ ID NO: 73, SEQ ID NO: 76, or SEQ ID NO: 77.

In some embodiments, the influenza virus is an influenza A virus. In some embodiments, the influenza virus is an influenza B virus. In some embodiments, the influenza virus is an influenza C virus.

### Definitions

To facilitate the understanding of this invention, a number of terms are defined below. Terms defined herein have meanings as commonly understood by a person of ordinary skill in the areas relevant to the present invention. Terms such as “a,” “an,” and “the” are not intended to refer to only a singular entity, but include the general class of which a specific example may be used for illustration. The terminology herein is used to describe specific embodiments of the invention, but their usage does not delimit the invention, except as outlined in the claims.

By “influenza infection” is meant the pathogenic growth of an influenza virus in a host organism (e.g., a human subject). An influenza infection can be any situation in which the presence of an influenza viral population damaging to a host body. Thus, a subject is “suffering” from an influenza infection when an excessive amount of an influenza population is present in or on the subject's body, or when the presence of a viral population(s) is damaging the cells or other tissue of the subject.

As used herein, the term “Fc domain monomer” refers to a polypeptide chain that includes at least a hinge domain and second and third antibody constant domains (C_{H}2 and C_{H}3) or functional fragments thereof (e.g., fragments that that capable of (i) dimerizing with another Fc domain monomer to form an Fc domain, and (ii) binding to an Fc receptor. The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b, IgG3, or IgG4) (e.g., IgG1). An Fc domain monomer does not include any portion of an immunoglobulin that is capable of acting as an antigen-recognition region, e.g., a variable domain or a complementarity determining region (CDR). Fc domain monomers in the conjugates as described herein can contain one or more changes from a wild-type Fc domain monomer sequence (e.g., 1-10, 1-8, 1-6, 1-4 amino acid substitutions, additions, or deletions) that alter the interaction between an Fc domain and an Fc receptor. Examples of suitable changes are known in the art. In certain embodiments, a human Fc domain monomer (e.g., an IgG heavy chain, such as IgG1) comprises a region that extends from any of Asn208, Glu216, Asp221, Lys222, or Cys226 to the carboxyl-terminus of the heavy chain at Lys447. C-terminal Lys447 of the Fc region may or may not be present, without affecting the structure or stability of the Fc region. C-terminal Lys 447 may be proteolytically cleaved upon expression of the polypeptide. In some embodiments of any of the Fc domain monomers described herein, C-terminal Lys 447 is optionally present or absent. The disclosure specifically contemplates any of SEQ ID NOs: 1-4, 11, 16, 19, 20, 32-37, 48-53, and 60-68 that do not include the C-terminal Lys corresponding to Lys447. The N-terminal N (Asn) of the Fc region (e.g., of any one of SEQ ID NOs: 60-77) may or may not be present, without affecting the structure of stability of the Fc region. N-terminal Asn may be deamidated upon expression of the polypeptide. In some embodiments of any of the Fc domain monomers described herein, N-terminal Asn is optionally present or absent. The disclosure specifically contemplates any of SEQ ID NOs: 60-77 that do not include the N-terminal Asn. Unless otherwise specified herein, numbering of amino acid residues in the IgG or Fc domain monomer is according to the EU numbering system for antibodies, also called the Kabat EU index, as described, for example, in Kabat et al., Sequences of Proteins of Immunological Interest, 5th Ed. Public Health Service, National Institutes of Health, Bethesda, MD, 1991.

As used herein, the term “Fc domain” refers to a dimer of two Fc domain monomers that is capable of binding an Fc receptor. In the wild-type Fc domain, the two Fc domain monomers dimerize by the interaction between the two C_{H}3 antibody constant domains, in some embodiments, one or more disulfide bonds form between the hinge domains of the two dimerizing Fc domain monomers.

As used-herein, a “surface exposed amino acid” or “solvent-exposed amino acid,” such as a surface exposed cysteine or a surface exposed lysine refers to an amino acid that is accessible to the solvent surrounding the protein. A surface exposed amino acid may be a naturally-occurring or an engineered variant (e.g., a substitution or insertion) of the protein. In some embodiments, a surface exposed amino acid is an amino acid that when substituted does not substantially change the three-dimensional structure of the protein.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat” a human subject having an influenza infection means to administering treatment to a subject already suffering from an influenza infection to improve or stabilize the subject's condition. Thus, in the claims and embodiments, treating is the administration to a subject either for therapeutic or prophylactic purposes.

The term “T,” as used herein, refers to the number of dimers of neuraminidase inhibitors conjugated to an Fc domain within a population of conjugates. In some embodiments, within a population of conjugates, the average number of dimers of neuraminidase inhibitors (i.e., the average (mean) value of T) conjugated to an Fc domain monomer may be from 1 to 20 (e.g., the average value of T is 3 to 6 or 3.5 to 5.5). In some embodiments, the average value of T is 4.5.

As used herein, the term “pharmaceutical composition” refers to a medicinal or pharmaceutical formulation that contains a conjugate of formula (I) and one or more excipients and diluents to enable the active ingredient suitable for the method of administration. The pharmaceutical composition of the present disclosure includes pharmaceutically acceptable components that are compatible with a conjugate of Formula (I).

As used herein, the term “pharmaceutically acceptable carrier” refers to an excipient or diluent in a pharmaceutical composition. For example, a pharmaceutically acceptable carrier may be a vehicle capable of suspending or dissolving a conjugate of Formula (I). The pharmaceutically acceptable carrier must be compatible with the other ingredients of the formulation and not deleterious to the recipient. In the present disclosure, the pharmaceutically acceptable carrier must provide adequate pharmaceutical stability to a conjugate described herein.

The term “pharmaceutically acceptable salt,” as used herein, represents salts of a conjugates of Formula (I) that are, within the scope of sound medical judgment, suitable for use in methods described herein without undue toxicity, irritation, and/or allergic response. Pharmaceutically acceptable salts are well known in the art. For example, pharmaceutically acceptable salts are described in: Pharmaceutical Salts: Properties, Selection, and Use (Eds. P. H. Stahl and C. G. Wermuth), Wiley-VCH, 2008. The salts can be prepared in situ during the final isolation and purification of the conjugates described herein or separately by reacting the free base group with a suitable organic acid.

The term “drug-to-antibody ratio” or “DAR” refers to the average number of small molecule drug moieties (e.g., the average number of small molecule drug monomers or dimers) conjugated to an Fc domain. In some embodiments described herein, the DAR is represented by “T” (e.g., in formula (I)). As used herein, each dimer moiety conjugated to the Fc domain corresponds to a DAR value of 1.0 (e.g., a “T” value of 1.0). For example, an Fc domain conjugated to four zanamivir dimers would have a DAR of 4.0 (e.g., a “T” of 4.0). DAR may also be computed as the average DAR for a population of molecules, such as a population of Fc domains. DAR values may affect the efficacy, potency, pharmacokinetics, or toxicity of the drug.

By “a conjugate of Formula (I)” is meant a conjugate having the structure:

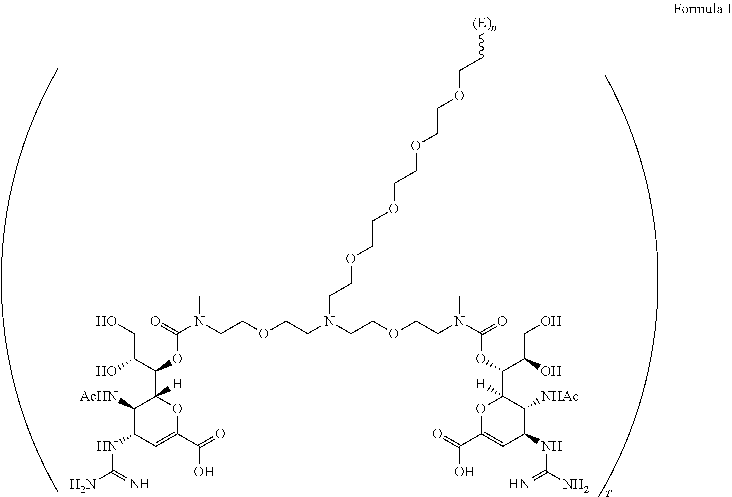

wherein each E includes an Fc domain monomer; n is 2; and T is an integer from 3 to 6; the squiggly line indicates a covalent attachment to a nitrogen atom of a solvent-exposed lysine or to a sulfur atom of a solvent-exposed cysteine of an E. In the conjugate, the conjugation of the zanamivir dimer is at various solvent exposed lysines on the Fc domain monomer.

By “Conjugate A” is meant a conjugate of Formula (I) in which the average value of T is about 4.5 and each E has the sequence of SEQ ID NO: 76.

### DESCRIPTION OF THE DRAWINGS

FIG. 1A is a summary of the study protocol of the clinical trial and FIG. 1B is a summary of the disposition of the clinical trial.

FIG. 2 is a summary of the demographics of the human subjects in the clinical trial.

FIG. 3 is a boxplot of area under the viral load-time curve (VL-AUC) data of placebo and Conjugate A.

FIG. 4 is a graph of the mean viral load from qRT-PCR over eight days of placebo and Conjugate A.

FIG. 5 is a boxplot of the peak viral load data of placebo and Conjugate A.

FIG. 6 is a graph showing the time to confirmed negative test of influenza by qRT-PCR of placebo and Conjugate A.

FIG. 7 is a boxplot of the area under the curve over time of total clinical symptoms score (TSS-AUC) of placebo and Conjugate A.

FIG. 8 is a graph of mean total clinical symptoms score (TSS) over time of placebo and Conjugate A.

FIG. 9 is a boxplot of peak total clinical symptoms score (TSS) of placebo and Conjugate A.

FIG. 10 is a graph of time to symptom resolution of placebo and Conjugate A.

FIG. 11 is a graph of single dose mean plasma Conjugate A concentrations versus time for 50 mg Conjugate A administered by intramuscular or subcutaneous injection.

FIG. 12 is a graph of single dose mean plasma Conjugate A concentrations versus time for 150 mg Conjugate A administered by intramuscular or subcutaneous injection.

FIG. 13 is a graph of single dose mean plasma Conjugate A concentrations versus time for 50 or 150 mg Conjugate A administered by intramuscular or subcutaneous injection on a semi-logarithmic scale.

FIG. 14 is a graph of single dose mean plasma Conjugate A concentrations versus time for 50, 150, or 450 mg Conjugate A administered by intramuscular or subcutaneous injection on a semi-logarithmic scale.

FIG. 15 is a graph showing the mean AUC of Conjugate A as a function of Conjugate A dose (mg) when Conjugate A is administered intramuscularly versus subcutaneously.

FIG. 16 is a graph showing the predicted median (line) and the 10^{th} and 90^{th} percentiles (shaded grey area) of Conjugate A concentrations relative to individual Cₘₐₓ. Day 5 is shown as dotted vertical line. Gray rectangle is the in-house period for participants in the human challenge study.

FIG. 17 is a graph showing model-predicted plasma concentrations of Conjugate A given as: a 150 mg dose once, a 300 mg dose once, or a 150 mg dose twice.

### DETAILED DESCRIPTION

### Influenza

The conjugates and pharmaceutical compositions described herein can be used to treat an influenza infection such as an infection of influenza A, B, or C.

Influenza viruses are associated with significant human disease and cause annual epidemics during autumn and winter. Although most people recover from seasonal influenza within 1 to 2 weeks without requiring medical attention, globally, millions are hospitalised each year and about 650,000 deaths occur due to influenza, particularly among the very young, elderly, and chronically ill. In the United States (US), an estimated 37.4-42.9 million symptomatic influenza-related illnesses, 17.3-20.1 million influenza-related medical visits, 531,000-647,000 influenza-related hospitalisations, and 63,400-61,200 deaths occurred during the 2018-2019 influenza season. Comparable mortality and morbidity rates have been reported for European countries. These numbers remain high year after year because currently no effective medicine is available for the prevention of influenza. Currently, the influenza type A viruses H1N1 and H3N2 are circulating in humans, along with influenza B viruses. H3N2 viruses have been predominant in most seasons and have caused a higher number of deaths and hospitalisations than H1N1 and influenza B viruses.

Progress towards a medicinal agent that provides protection against a broad range of influenza strains with a longer duration of protection, otherwise known as a “universal vaccine”, has been disappointing. The monoclonal antibody therapeutics developed to date have suffered from limited spectrum and commercial limitations due to high dosing requirements and/or the need for multiple antibody cocktails to achieve a desired spectrum and efficacy. Thus, a significant unmet need exists for long-acting universal protective agents.

The influenza human challenge model was established to not only aid understanding of influenza disease and transmission, but to also assess the efficacy of antivirals, immunomodulators, and vaccines. The influenza H3N2 A/Perth/16/2009 challenge strain has been used in the majority of studies to date. The challenge virus has been shown to induce measurable disease profiles with clear distinction from non-infected participants and study participants have approximately 60% to 75% chance of becoming infected following the administration of the virus. Typical influenza illness is characterized by an abrupt onset of rhinitis, nasal stuffiness, fever, malaise, myalgia (muscle aches), and sore throat. In healthy adults, the illness usually resolves without any treatment, with relief of symptoms occurring naturally within 3 to 5 days. The disease profiles of the challenge agent are consistent with the mild to moderate disease profiles expected with wild-type challenge viruses in healthy adult participants. In summary, the influenza H3N2 A/Perth/16/2009 challenge virus is considered safe, well tolerated, and induces appropriate disease pathogenesis to be an effective viral challenge agent in the human viral challenge (HVC) studies.

### Conjugate A—A Long-acting Antiviral Fc-Conjugate

Conjugate A is a zanamivir-crystallisable fragment (Fc) conjugate. Zanamivir is a small molecule antiviral neuraminidase inhibitor. The small molecule targeting groups selectively engage a small, conserved pocket on the viral surface, which is not feasible with monoclonal antibodies.

Conjugate A differs from traditional antibody-drug conjugate molecules in the following aspects:
in traditional antibody-drug conjugates, the drug is attached to full length human IgG (Fc plus the antigen-binding fragment); in contrast, zanamivir dimers of Conjugate A are conjugated to an Fc fragment of human IgG1 (and not full length IgG1). In the conjugate, the conjugation of the zanamivir dimer is at a different solvent exposed lysine on each Fc domain monomer. In some embodiments, the zanamivir dimer is conjugated to one or more solvent-exposed lysines, e.g., Lys205, Lys213, Lys218, Lys246, Lys317, Lys326, Lys334, Lys392, or a combination thereof.

In traditional antibody-drug conjugates, the drug is conjugated to the human IgG using a protease-cleavable linker to allow release of the drug inside target cells; the linker between zanamivir and the Fc in Conjugate A is not a substrate for proteases, and it exerts its antiviral activity in the extracellular space.

Traditional antibody-drug conjugates are used to treat cancer by delivering cytotoxic payloads to target cells with rapid release; Conjugate A is designed to treat and prevent infectious disease using a long-acting stable conjugate of a non-cytotoxic small molecule to an Fc fragment of IgG1.

Methods for making Conjugate A are described in U.S. Pat. No. 11,510,992, hereby incorporated by reference in its entirety.

### Fc Domains

Fc domains are dimers of two Fc domain monomers, which generally domains include a hinge domain, a C_{H}2 antibody constant domain, and a C_{H}3 antibody constant domain. The Fc domain monomer can be of immunoglobulin antibody isotype IgG, IgE, IgM, IgA, or IgD. An Fc domain monomer can be of any immunoglobulin antibody isotype (e.g., IgG1, IgG2a, IgG2b, IgG3, or IgG4). The Fc domain monomer can be of any immunoglobulin antibody allotype (e.g., IGHG1*01 (i.e., G1m(za)), IGHG1*07 (i.e., G1m (zax)), IGHG1*04 (i.e., G1m(zav)), IGHG1*03 (G1m(f)), IGHG1*08 (i.e., G1m(fa)), IGHG2*01, IGHG2*06, IGHG2*02, IGHG3*01, IGHG3*05, IGHG3*10, IGHG3*04, IGHG3*09, IGHG3*11, IGHG3*12, IGHG3*06, IGHG3*07, IGHG3*08, IGHG3*13, IGHG3*03, IGHG3*14, IGHG3*15, IGHG3*16, IGHG3*17, IGHG3*18, IGHG3*19, IGHG2*04, IGHG4*01, IGHG4*03, or IGHG4*02) (as described in, for example, in Vidarsson et al. IgG subclasses and allotypes: from structure to effector function. Frontiers in Immunology. 5 (520): 1-17 (2014)). The Fc domain monomer can also be of any species, e.g., human, murine, or mouse. A dimer of Fc domain monomers (i.e., an Fc domain) can bind to an Fc receptor, which is a receptor located on the surface of leukocytes.

In some embodiments, an Fc domain monomer in the conjugates described herein may contain one or more amino acid substitutions, additions, and/or deletion relative to an Fc domain monomer having a sequence of any one of SEQ ID NOs: 1-138. In some embodiments, an Asn in an Fc domain monomer in the conjugates as described herein may be replaced by Ala in order to prevent N-linked glycosylation (see, e.g., SEQ ID NOs: 12-15, where Asn to Ala substitution is labeled with *). In some embodiments, an Fc domain monomer in the conjugates described herein may also containing additional Cys additions (see, e.g., SEQ ID NOs: 9, 10, and 11, where Cys additions are labeled with *).

In some embodiments, an Fc domain monomer in the conjugates as described herein includes an additional moiety, e.g., an albumin-binding peptide, a purification peptide (e.g., a hexa-histidine peptide (HHHHHH (SEQ ID NO: 146)), or a signal sequence (e.g., IL2 signal sequence MYRMQLLSCIALSLALVTNS (SEQ ID NO: 147)) attached to the N- or C-terminus of the Fc domain monomer. In some embodiments, an Fc domain monomer in the conjugate does not contain any type of antibody variable region, e.g., V_{H}, V_{L}, a complementarity determining region (CDR), or a hypervariable region (HVR).

In some embodiments, an Fc domain monomer in the conjugates as described herein may have a sequence that is at least 95% identical (e.g., 97%, 99%, or 99.5% identical) to the sequence of any one of SEQ ID NOs: 1-138 shown below. In some embodiments, an Fc domain monomer in the conjugates as described herein may have a sequence of any one of SEQ ID NOs: 1-138 shown below.

SEQ ID NO: 1: murine Fc-IgG2a with IL2 signal sequence at the N-terminus (bold)
MYRMQLLSCIALSLALVTNSPRGPTIKPCPPCKCPAPNLLGGPSVFIFPPKIKDVLMISLSPIVTCVVVD
VSEDDPDVQISWFVNNVEVHTAQTQTHREDYNSTLRVVSALPIQHQDWMSGKEFKCKVNNKDLPAP
IERTISKPKGSVRAPQVYVLPPPEEEMTKKQVTLTCMVTDFMPEDIYVEWTNNGKTELNYKNTEPVLD
SDGSYFMYSKLRVEKKNWVERNSYSCSVVHEGLHNHHTTKSFSRTPGK
SEQ ID NO: 2: mature murine Fc-IgG2a
PRGPTIKPCPPCKCPAPNLLGGPSVFIFPPKIKDVLMISLSPIVTCVVVDVSEDDPDVQISWFVNNVEV
HTAQTQTHREDYNSTLRVVSALPIQHQDWMSGKEFKCKVNNKDLPAPIERTISKPKGSVRAPQVYVL
PPPEEEMTKKQVTLTCMVTDFMPEDIYVEWTNNGKTELNYKNTEPVLDSDGSYFMYSKLRVEKKNW
VERNSYSCSVVHEGLHNHHTTKSFSRTPGK
SEQ ID NO: 3: human Fc-IgG1 with IL2 signal sequence at the N-terminus (bold) and N-terminal
MVRS amino acid residues added (underlined)
MYRMQLLSCIALSLALVTNSMVRSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 4: mature human Fc-IgG1 with N-terminal MVRS amino acid residues added (underlined)
MVRSDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGK
SEQ ID NO: 5: murine Fc-IgG2a with IL2 signal sequence (bold) at the N-terminus and hexa-histidine
peptide (italicized) at the C-terminus
MYRMQLLSCIALSLALVTNSPRGPTIKPCPPCKCPAPNLLGGPSVFIFPPKIKDVLMISLSPIVTCVVVD
VSEDDPDVQISWFVNNVEVHTAQTQTHREDYNSTLRVVSALPIQHQDWMSGKEFKCKVNNKDLPAP
IERTISKPKGSVRAPQVYVLPPPEEEMTKKQVTLTCMVTDFMPEDIYVEWTNNGKTELNYKNTEPVLD
SDGSYFMYSKLRVEKKNWVERNSYSCSVVHEGLHNHHTTKSFSRTPGKHHHHHH
SEQ ID NO: 6: mature murine Fc-IgG2a with hexa-histidine peptide (italicized) at the C-terminus
PRGPTIKPCPPCKCPAPNLLGGPSVFIFPPKIKDVLMISLSPIVTCVVVDVSEDDPDVQISWFVNNVEV
HTAQTQTHREDYNSTLRVVSALPIQHQDWMSGKEFKCKVNNKDLPAPIERTISKPKGSVRAPQVYVL
PPPEEEMTKKQVTLTCMVTDFMPEDIYVEWTNNGKTELNYKNTEPVLDSDGSYFMYSKLRVEKKNW
VERNSYSCSVVHEGLHNHHTTKSFSRTPGKHHHHHH
SEQ ID NO: 7: human Fc-IgG1 with IL2 signal sequence (bold) at the N-terminus, N-terminal MVRS
amino acid residues added (underlined), and hexa-histidine peptide (italicized) at the C-terminus
MYRMQLLSCIALSLALVTNSMVRSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGKHHHHHH
SEQ ID NO: 8: mature human Fc-IgG1 with hexa-histidine peptide (italicized) at the C-terminus and
N-terminal MVRS amino acid residues added (underlined)
MVRSDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGKHHHHHH
SEQ ID NO: 9: human Fc-IgG1 with IL2 signal sequence (bold) at the N-terminus, N-terminal MVRS
amino acid residues added (underlined), two additional cysteines in the hinge region (*), and hexa-
histidine peptide (italicized) at the C-terminus
MYRMQLLSCIALSLALVTNSMVRSDKTHTCPPCPPC*KC*PAPELLGGPSVFLFPPKPKDTLMISRTP
EVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGKHHHHHH
SEQ ID NO: 10: mature human Fc-IgG1 with N-terminal MVRS amino acid residues added
(underlined), two additional cysteines in the hinge region (*), and hexa-histidine peptide
(italicized) at the C-terminus
MVRSDKTHTCPPCPPC*KC*PAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGKHHHHHH
SEQ ID NO: 11: mature human Fc-IgG1 with N-terminal MVRS amino acid residues added
(underlined) and two additional cysteines in the hinge region (*)
MVRSDKTHTCPPCPPC*KC*PAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 12: murine Fc-IgG2a with IL2 signal sequence (bold) at the N-terminus, Asn to Ala
substitution (*), and hexa-histidine peptide (italicized) at the C-terminus
MYRMQLLSCIALSLALVTNSPRGPTIKPCPPCKCPAPNLLGGPSVFIFPPKIKDVLMISLSPIVTCVVVD
VSEDDPDVQISWFVNNVEVHTAQTQTHREDYA*STLRVVSALPIQHQDWMSGKEFKCKVNNKDLPA
PIERTISKPKGSVRAPQVYVLPPPEEEMTKKQVTLTCMVTDFMPEDIYVEWTNNGKTELNYKNTEPVL
DSDGSYFMYSKLRVEKKNWVERNSYSCSVVHEGLHNHHTTKSFSRTPGKHHHHHH
SEQ ID NO: 13: mature murine Fc-IgG2a with Asn to Ala substitution (*) and hexa-histidine peptide
(italicized) at the C-terminus
PRGPTIKPCPPCKCPAPNLLGGPSVFIFPPKIKDVLMISLSPIVTCVVVDVSEDDPDVQISWFVNNVEV
HTAQTQTHREDYA*STLRVVSALPIQHQDWMSGKEFKCKVNNKDLPAPIERTISKPKGSVRAPQVYVL
PPPEEEMTKKQVTLTCMVTDFMPEDIYVEWTNNGKTELNYKNTEPVLDSDGSYFMYSKLRVEKKNW
VERNSYSCSVVHEGLHNHHTTKSFSRTPGKHHHHHH
SEQ ID NO: 14: human Fc-IgG1 with IL2 signal sequence (bold) at the N-terminus, N-terminal MVRS
amino acid residues added (underlined), Asn to Ala substitution (*), and hexa-histidine peptide
(italicized) at the C-terminus
MYRMQLLSCIALSLALVTNSMVRSDKTHTCPPCPAPELLGGPSVFLFPPKPKDTLMISRTPEVTCVVV
DVSHEDPEVKFNWYVDGVEVHNAKTKPREEQYA*STYRVVSVLTVLHQDWLNGKEYKCKVSNKALP
APIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGKHHHHHH
SEQ ID NO: 15: mature human Fc-IgG1 with Asn to Ala substitution (*), N-terminal MVRS amino acid
residues added (underlined), and hexa-histidine peptide (italicized) at the C-terminus
MVRSDKTHTCPPCPAPELLGGPSVFLFPPKPKDTLMISRTPEVTCVVVDVSHEDPEVKFNWYVDGVE
VHNAKTKPREEQYA*STYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGKHHHHHH
SEQ ID NO: 16: human IgG1 Fc with Human Serum Albumin Signal Sequence (bold) at the N-
terminus and N-terminal ISAMVRS amino acid residues added (underlined)
MKWVTFISLLFLFSSAYSISAMVRSDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
SEQ ID NO: 17: human IgG1 Fc with Human Serum Albumin Signal Sequence (bold) at the N-
terminus, N-terminal ISAMVRS amino acid residues added (underlined), C-terminal G4S linker
(italicized), and C-terminal c-Myc tag (underlined, italicized)
MKWVTFISLLFLFSSAYSISAMVRSDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGGGGGSEQKLISEEDL
SEQ ID NO: 18: mature human IgG1 Fc with N-terminal ISAMVRS amino acid residues added
(underlined), C-terminal G4S linker (italicized), and C-terminal c-Myc tag (underlined, italicized)
ISAMVRSDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGGGGGSEQKLISEEDL
SEQ ID NO: 19: human IgG1 Fc with Human Serum Albumin Signal Sequence (bold), N-terminal
ISAMVRS amino acid residues added (underlined), and lysine to serine modification (*) to prevent
lysine conjugation at this site
MKWVTFISLLFLFSSAYSISAMVRSDKTHTCPPCPAPELLGGPSVFLFPPKPS*DTLMISRTPEVTCVV
VDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
SEQ ID NO: 20: mature human IgG1 Fc with N-terminal ISAMVRS amino acid residues added
(underlined) and lysine to serine modification (*) to prevent lysine conjugation at this site
ISAMVRSDKTHTCPPCPAPELLGGPSVFLFPPKPS*DTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
SEQ ID NO: 21: human IgG1 Fc with Human Serum Albumin Signal Sequence (bold) at the N-
terminus, N-terminal ISAMVRS amino acid residues added (underlined), lysine to serine modification
(*) to prevent lysine conjugation at this site, C-terminal G4S linker (italicized), and C-terminal C-Myc
tag (underlined, italicized)
MKWVTFISLLFLFSSAYSISAMVRSDKTHTCPPCPAPELLGGPSVFLFPPKPS(*)DTLMISRTPEVTCV
VVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGGGGGSEQKLISEEDL
SEQ ID NO: 22: mature human IgG1 Fc with N-terminal ISAMVRS amino acid residues added
(underlined), lysine to serine modification (*) to prevent lysine conjugation at this site, C-terminal
G4S linker (italicized), and C-terminal C-Myc tag (underlined, italicized)
ISAMVRSDKTHTCPPCPAPELLGGPSVFLFPPKPS(*)DTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGGGGGSEQKLISEEDL
SEQ ID NO: 23: human IgG1 Fc with Human Serum Albumin Signal Sequence (bold) at the N-
terminus, N-terminal ISAMVRS amino acid residues added (underlined), Asn to Ala substitution (*), C-
terminal G4S linker (italicized), and C-terminal C-myc tag (underlined, italicized)
MKWVTFISLLFLFSSAYSISAMVRSDKTHTCPPCPAPELLGGPSVFLFPPKPKDTLMISRTPEVTCVV
VDVSHEDPEVKFNWYVDGVEVHNAKTKPREEQYA(*)STYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGGGGGSEQKLISEEDL
SEQ ID NO: 24: mature human IgG1 Fc with N-terminal ISAMVRS amino acid residues added
(underlined), Asn to Ala substitution (*), C-terminal G4S linker (italicized), and C-terminal C-myc tag
(underlined, italicized)
ISAMVRSDKTHTCPPCPAPELLGGPSVFLFPPKPKDTLMISRTPEVTCVVVDVSHEDPEVKFNWYVD
GVEVHNAKTKPREEQYA(*)STYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGGGGGSEQKLISEEDL
SEQ ID NO: 25: human IgG1 Fc with Human Serum Albumin Signal Sequence (bold) at the N-
terminus, N-terminal ISAMVRS amino acid residues added (underlined), H310A (*) and H435A (*)
mutations to impede FcRn binding, C-terminal G4S (italicized), and C-terminal C-myc tag (underlined,
italicized)
MKWVTFISLLFLFSSAYSISAMVRSDKTHTCPPCPAPELLGGPSVFLFPPKPKDTLMISRTPEVTCVV
VDVSHEDPEVKFNWYVDGVEVHNAKTKPREEQYNSTYRVVSVLTVLA(*)QDWLNGKEYKCKVSNKA
LPAPIEKTISKA(*)KGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNAYTQKSLSLSPGGGGGSEQKLISEE
DL
SEQ ID NO: 26: mature human IgG1 Fc with Human Serum Albumin Signal Sequence (bold) at the
N-terminus, N-terminal ISAMVRS amino acid residues added (underlined), with H310A (*) and H435A
(*) mutations to impede FcRn binding, C-terminal G4S (italicized), and C-terminal C-myc tag
(underlined, italicized)
ISAMVRSDKTHTCPPCPAPELLGGPSVFLFPPKPKDTLMISRTPEVTCVVVDVSHEDPEVKFNWYVD
GVEVHNAKTKPREEQYNSTYRVVSVLTVLA(*)QDWLNGKEYKCKVSNKALPAPIEKTISKA(*)KGQPR
EPQVYTLPPSREEMTKNQVSLTCLVKGFYPSDIAVEWESNGQPENNYKTTPPVLDSDGSFFLYSKLT
VDKSRWQQGNVFSCSVMHEALHNAYTQKSLSLSPGGGGGSEQKLISEEDL
SEQ ID NO: 27: human IgG1 Fc with Human Serum Albumin Signal Sequence (bold) at the N-
terminus, N-terminal ISAMVRS amino acid residues added (underlined), C-terminal G4S linker
(italicized), and C-terminal mutated (lysine to phenylalanine, bold) C-myc tag (underlined, italicized)
MKWVTFISLLFLFSSAYSISAMVRSDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGGGGGSEQFLISEEDL
SEQ ID NO: 28: mature human IgG1 Fc with N-terminal ISAMVRS amino acid residues added
(underlined), C-terminal G4S linker (italicized), and C-terminal mutated (lysine to phenylalanine, bold)
C-myc tag (underlined, italicized)
ISAMVRSDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGGGGGSEQFLISEEDL
SEQ ID NO: 29: human IgG1 Fc with Human Serum Albumin Signal Sequence (bold) at the N-
terminus, N-terminal ISAMVRS amino acid residues added (underlined), Asn to Ala substitution (*), C-
terminal G4S linker (italicized), and C-terminal mutated (lysine to phenylalanine, bold) C-myc tag
(underlined, italicized)
MKWVTFISLLFLFSSAYSISAMVRSDKTHTCPPCPAPELLGGPSVFLFPPKPKDTLMISRTPEVTCVV
VDVSHEDPEVKFNWYVDGVEVHNAKTKPREEQYA(*)STYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGGGGGSEQFLISEEDL
SEQ ID NO: 30: mature human IgG1 Fc with N-terminal MVRS amino acid residues added
(underlined), Asn to Ala substitution (*), C-terminal G4S linker (italicized), and C-terminal mutated
(lysine to phenylalanine, bold) C-myc tag (underlined, italicized)
ISAMVRSDKTHTCPPCPAPELLGGPSVFLFPPKPKDTLMISRTPEVTCVVVDVSHEDPEVKFNWYVD
GVEVHNAKTKPREEQYA(*)STYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGGGGGSEQFLISEEDL
SEQ ID NO: 31: human IgG1 Fc with Human Serum Albumin Signal Sequence (bold) at the N-
terminus, allotype G1m(fa) (bold italics), C-terminal G4S linker (italicized), and C-terminal mutated
(lysine to phenylalanine, bold) C-myc tag (underlined)
MKWVTFISLLFLFSSAYSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGGGGGSEQFLISEEDL
SEQ ID NO: 32: human IgG1 Fc with Human Serum Albumin Signal Sequence (bold) at the N-
terminus, allotype G1m(fa) (bold italics)
MKWVTFISLLFLFSSAYSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 33: mature human IgG1 Fc with a YTE triple mutation (bold and underlined) with N-
terminal MVRS amino acid residues added (underlined)
MVRSDKTHTCPPCPAPELLGGPSVFLFPPKPKDTLYITREPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGK
SEQ ID NO: 34: human IgG1 Fc with Human Serum Albumin Signal Sequence (bold) at the N-
terminus, contains residues EPKSS comprising the full hinge region on the N-terminus of mature
human IgG1 Fc (underlined), Cys to Ser substitution (#), allotype G1m(fa) (bold italics)
MKWVTFISLLFLFSSAYSEPKSS(#)DKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK
SEQ ID NO: 35: human IgG1 Fc with murine IgG signal sequence (bold) at the N-terminus, with
removal of EPKSSD hinge residues from the N-terminus of the mature human IgG1 Fc, allotype
G1m(fa) (bold italics)
MGWSCIILFLVATATGVHSKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 36: mature human IgG1 Fc with a YTE triple mutation (bold and underlined), with
removal of EPKSSD hinge residues from the N-terminus of the mature human IgG1 Fc, allotype
G1m(fa) (bold italics)
KTHTCPPCPAPELLGGPSVFLFPPKPKDTLYITREPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVF
SCSVMHEALHNHYTQKSLSLSPGK
SEQ ID NO: 37: mature human IgG1 Fc with an LS double mutation (bold and underlined), with
removal of EPKSSD hinge residues from the N-terminus of the mature human IgG1 Fc, allotype
G1m(fa) (bold italics)
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVF
SCSVLHEALHSHYTQKSLSLSPGK
SEQ ID NO: 38: mature human IgG1 Fc with Human Serum Albumin Signal Sequence (bold) at the
N-terminus, a YTE triple mutation (bold and underlined), allotype G1m(fa) (bold italics), C-terminal
G4S linker (italicized), and C-terminal C-myc tag (underlined)
MKWVTFISLLFLFSSAYSDKTHTCPPCPAPELLGGPSVFLFPPKPKDTLYITREPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGGGGGSEQKLISEEDL
SEQ ID NO: 39: mature human Fc IgG1, wherein X₁ is Met or Tyr, X₂ is Ser or Thr, X₃ is Thr or Glu,
X₄ is Asp or Glu, and X₅ is Leu or Met, X₆ is Met or Leu, and X₇ is Asn or Ser
DKTHTCPPCPAPELLGGPSVFLFPPKPKDTLX₁IX₂RX₃PEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLP
PSRX₄EX₅TKNQVSLTCLVKGFYPSDIAVEWESNGQPENNYKTTPPVLDSDGSFFLYSKLTVDKSRW
QQGNVFSCSVX₆HEALHX₇HYTQKSLSLSPG
SEQ ID NO: 40: mature human Fc IgG1 wherein X₄ is Asp or Glu, and X₅ is Leu or Met
DKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
RX₄EX₅TKNQVSLTCLVKGFYPSDIAVEWESNGQPENNYKTTPPVLDSDGSFFLYSKLTVDKSRWQQ
GNVFSCSVMHEALHNHYTQKSLSLSPG
SEQ ID NO: 41: mature human Fc IgG1 with a YTE triple mutation (bold and underlined), and wherein
X₄ is Asp or Glu, and X₅ is Leu or Met
DKTHTCPPCPAPELLGGPSVFLFPPKPKDTLYITREPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
RX₄EX₅TKNQVSLTCLVKGFYPSDIAVEWESNGQPENNYKTTPPVLDSDGSFFLYSKLTVDKSRWQQ
GNVFSCSVMHEALHNHYTQKSLSLSPG
SEQ ID NO: 42: mature human Fc IgG1 with a YTE triple mutation (bold and underlined), allotype
G1m(fa) (bold italics)
DKTHTCPPCPAPELLGGPSVFLFPPKPKDTLYITREPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQG
NVFSCSVMHEALHNHYTQKSLSLSPG
SEQ ID NO: 43: mature human Fc IgG1 with a YTE triple mutation (bold and underlined), allotype
G1m(f) (bold italics)
DKTHTCPPCPAPELLGGPSVFLFPPKPKDTLYITREPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVDKSRWQQG
NVFSCSVMHEALHNHYTQKSLSLSPG
SEQ ID NO: 44: mature human Fc IgG1 with a LS double mutation (bold and underlined), and
wherein X₄ is Asp or Glu, and X₅ is Leu or Met
DKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
RX₄EX₅TKNQVSLTCLVKGFYPSDIAVEWESNGQPENNYKTTPPVLDSDGSFFLYSKLTVDKSRWQQ
GNVFSCSVLHEALHSHYTQKSLSLSPG
SEQ ID NO: 45: mature human Fc IgG1 with a LS double mutation (bold and underlined), allotype
G1m(fa) (bold italics)
DKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQG
NVFSCSVLHEALHSHYTQKSLSLSPG
SEQ ID NO: 46: mature human Fc IgG1 with a LS double mutation (bold and underlined), allotype
G1m(f) (bold italics)
DKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVDKSRWQQG
NVFSCSVLHEALHSHYTQKSLSLSPG
SEQ ID NO: 47: mature human Fc IgG1 with mouse heavy chain MIgG Vh signal sequence (bold),
Cys to Ser substitution (#), and wherein X₁ is Met or Tyr, X₂ is Ser or Thr, X₃ is Thr or Glu,
X₄ is Asp or Glu, and X₅ is Leu or Met, X₆ is Met or Leu, and X₇ is Asn or Ser
MGWSCIILFLVATATGVHSNVNHKPSNTKVDKKVEPKSS(#)DKTHTCPPCPAPELLGGPSVFLFPPK
PKDTLX₁IX₂RX₃PEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRX₄EX₅TKNQVSLTCLVKGFYPSDIA
VEWESNGQPENNYKTTPPVLDSDGSFFLYSKLTVDKSRWQQGNVFSCSVX₆HEALHX₇HYTQKSLSL
SPG
SEQ ID NO: 48: mature human IgG1 Fc with mouse heavy chain MIgG Vh signal sequence (bold),
Cys to Ser substitution (#), allotype G1m(fa) (bold italics)
MGWSCIILFLVATATGVHSNVNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GK
SEQ ID NO: 49: mature human IgG1 Fc with mouse heavy chain MIgG Vh signal sequence (bold),
Cys to Ser substitution (#), allotype G1m(f) (bold italics)
MGWSCIILFLVATATGVHSNVNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSP
GK
SEQ ID NO: 50: mature human IgG1 Fc with mouse heavy chain MIgG Vh signal sequence (bold),
Cys to Ser substitution (#), M428L, N434S mutations (Bold/Underlined), allotype G1m(fa) (bold
italics)
MGWSCIILFLVATATGVHSNVNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVLHEALHSHYTQKSLSLSPG
K
SEQ ID NO: 51: mature human IgG1 Fc with mouse heavy chain MIgG Vh signal sequence (bold),
Cys to Ser substitution (#), M428L, N434S mutations (Bold/Underlined), allotype G1m(f) (bold italics)
MGWSCIILFLVATATGVHSNVNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVLHEALHSHYTQKSLSLSPG
K
SEQ ID NO: 52: mature human IgG1 Fc with mouse heavy chain MIgG Vh signal sequence (bold),
Cys to Ser substitution (#), YTE triple mutation (bold and underlined), allotype G1m(fa) (bold italics)
MGWSCIILFLVATATGVHSNVNHKPSNTKVDKKVEPKSS(#)DKTHTCPPCPAPELLGGPSVFLFPPK
PKDTLYITREPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GK
SEQ ID NO: 53: mature human IgG1 Fc with mouse heavy chain MIgG Vh signal sequence (bold),
Cys to Ser substitution (#), YTE triple mutation (bold and underlined), allotype G1m(f) (bold italics)
MGWSCIILFLVATATGVHSNVNHKPSNTKVDKKVEPKSS(#)DKTHTCPPCPAPELLGGPSVFLFPPK
PKDTLYITREPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSP
GK
SEQ ID NO: 54: mature human IgG1 Fc with mouse heavy chain MIgG Vh signal sequence (bold), N-
terminal ISAMVRS amino acid residues added (italicized), M428L, N434S mutations
(bold/underlined), G4S linker (italicized), and C-terminal C-myc-tag (underlined), allotype G1m(f)
(bold italics)
MGWSCIILFLVATATGVHSISAMVRSDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVLHEALHSHYTQKSLSLSPGGGGGSEQKLISEEDL
SEQ ID NO: 55: mature human IgG1 Fc with mouse heavy chain MIgG Vh signal sequence (bold), N-
terminal ISAMVRS amino acid residues added (italicized), M428L, N434S mutations
(bold/underlined), G4S linker (italicized), C-terminal C-myc-tag (underlined), allotype G1m(fa)
(bold italics)
MGWSCIILFLVATATGVHSISAMVRSDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTP
PVLDSDGSFFLYSKLTVDKSRWQQGNVFSCSVLHEALHSHYTQKSLSLSPGGGGGSEQKLISEEDL
SEQ ID NO: 56: mature human IgG1 Fc with mouse heavy chain MIgG Vh signal sequence (bold), N-
terminal ISAMVRS amino acid residues added (italicized), YTE triple mutant (bold/underlined), G4S
linker (italicized), and C-terminal C-myc-tag (underlined), allotype G1m(f) (bold italics)
MGWSCIILFLVATATGVHSISAMVRSDKTHTCPPCPAPELLGGPSVFLFPPKPKDTLYITREPEVTCVV
VDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGGGGGSEQKLISEEDL
SEQ ID NO: 57: mature human IgG1 Fc with mouse heavy chain MIgG Vh signal sequence (bold), N-
terminal ISAMVRS amino acid residues added (italicized), YTE triple mutant (bold/underlined), G4S
linker (italicized), C-terminal C-myc-tag (underlined), allotype G1m(fa) (bold italics)
MGWSCIILFLVATATGVHSISAMVRSDKTHTCPPCPAPELLGGPSVFLFPPKPKDTLYITREPEVTCVV
VDVSHEDPEVKFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGGGGGSEQKLISEEDL
SEQ ID NO: 58: mature human IgG1 with mouse heavy chain MIgG1 signal sequence (bold), Cys to
Ser substitution (#), C-terminal G4S (italics), and C-terminal IgA peptide (underline), allotype
G1m(fa) (bold italics)
MGWSCIILFLVATATGVHSEPKSS(#)DKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGGGGGSQRNPRLRLIR
RHPTLRIPPI
SEQ ID NO: 59: mature human IgG1 with mouse heavy chain MIgG1 signal sequence (bold), Cys to
Ser substitution (#), M428L, N434S mutations (bold/underlined), C-terminal G4S (italics), and C-
terminal IgA peptide (underline), allotype G1m(fa) (bold italics)
MGWSCIILFLVATATGVHSEPKSS(#)DKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTP
PVLDSDGSFFLYSKLTVDKSRWQQGNVFSCSVLHEALHSHYTQKSLSLSPGGGGGSQRNPRLRLIR
RHPTLRIPPI
SEQ ID NO: 60: mature human Fc IgG1, Z₁ is Cys or Ser, and wherein X₁ is Met or Tyr, X₂ is Ser or
Thr, X₃ is Thr or Glu, X₄ is Asp or Glu, and X₅ is Leu or Met, X₆ is Met or Leu, and X₇ is Asn or Ser
NVNHKPSNTKVDKKVEPKSZ₁DKTHTCPPCPAPELLGGPSVFLFPPKPKDTLX₁IX₂RX₃PEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRX₄EX₅TKNQVSLTCLVKGFYPSDIAVEWESNGQPENNYKTTPPVL
DSDGSFFLYSKLTVDKSRWQQGNVFSCSVX₆HEALHX₇HYTQKSLSLSPGK
SEQ ID NO: 61: mature human Fc IgG1, Cys to Ser substitution (#), and wherein X₁ is Met or Tyr, X₂
is Ser or Thr, X₃ is Thr or Glu, X₄ is Asp or Glu, and X₅ is Leu or Met, X₆ is Met or Leu, and X₇
is Asn or Ser
NVNHKPSNTKVDKKVEPKSS(#)DKTHTCPPCPAPELLGGPSVFLFPPKPKDTLX₁IX₂RX₃PEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSRX₄EX₅TKNQVSLTCLVKGFYPSDIAVEWESNGQPENNYKTTPP
VLDSDGSFFLYSKLTVDKSRWQQGNVFSCSVX₆HEALHX₇HYTQKSLSLSPGK
SEQ ID NO: 62: mature human IgG1 Fc, Cys to Ser substitution (#), X₄ is Asp or Glu, and X₅ is Leu or
Met
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRX₄EX₅TKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 63: mature human IgG1 Fc, Cys to Ser substitution (#), allotype G1m(f) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 64: mature human IgG1 Fc, Cys to Ser substitution (#), allotype G1m(fa) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 65: mature human IgG1 Fc, Cys to Ser substitution (#), M428L, N434S mutations
(Bold/Underlined), allotype G1m(fa) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVLHEALHSHYTQKSLSLSPGK
SEQ ID NO: 66: mature human IgG1 Fc, Cys to Ser substitution (#), M428L, N434S mutations
(Bold/Underlined), allotype G1m(f) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVLHEALHSHYTQKSLSLSPGK
SEQ ID NO: 67: mature human IgG1 Fc, Cys to Ser substitution (#), YTE triple mutation (bold and
underlined), allotype G1m(fa) (bold italics)
NVNHKPSNTKVDKKVEPKSS(#)DKTHTCPPCPAPELLGGPSVFLFPPKPKDTLYITREPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 68: mature human IgG1 Fc, Cys to Ser substitution (#), YTE triple mutation (bold and
underlined), allotype G1m(f) (bold italics)
NVNHKPSNTKVDKKVEPKSS(#)DKTHTCPPCPAPELLGGPSVFLFPPKPKDTLYITREPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 69: mature human Fc IgG1, Z₁ is Cys or Ser, and wherein X₁ is Met or Tyr, X₂ is Ser or
Thr, X₃ is Thr or Glu, X₄ is Asp or Glu, and X₅ is Leu or Met, X₆ is Met or Leu, and X₇ is Asn or Ser
NVNHKPSNTKVDKKVEPKSZ₁DKTHTCPPCPAPELLGGPSVFLFPPKPKDTLX₁IX₂RX₃PEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRX₄EX₅TKNQVSLTCLVKGFYPSDIAVEWESNGQPENNYKTTPPVL
DSDGSFFLYSKLTVDKSRWQQGNVFSCSVX₆HEALHX₇HYTQKSLSLSPG
SEQ ID NO: 70: mature human Fc IgG1, Cys to Ser substitution (#), and wherein X₁ is Met or Tyr, X₂
is Ser or Thr, X₃ is Thr or Glu, X₄ is Asp or Glu, and X₅ is Leu or Met, X₆ is Met or Leu, and X₇
is Asn or Ser
NVNHKPSNTKVDKKVEPKSS(#)DKTHTCPPCPAPELLGGPSVFLFPPKPKDTLX₁IX₂RX₃PEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSRX₄EX₅TKNQVSLTCLVKGFYPSDIAVEWESNGQPENNYKTTPP
VLDSDGSFFLYSKLTVDKSRWQQGNVFSCSVX₆HEALHX₇HYTQKSLSLSPG
SEQ ID NO: 71: mature human IgG1 Fc, Cys to Ser substitution (#), X₄ is Asp or Glu, and X₅ is Leu or
Met
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRX₄EX₅TKNQVSLTCLVKGFYPSDIAVEWESNGQPENNYKTTPPVL
DSDGSFFLYSKLTVDKSRWQQGNVFSCSVMHEALHNHYTQKSLSLSPG
SEQ ID NO: 72: mature human IgG1 Fc, Cys to Ser substitution (#), allotype G1m(f) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
SEQ ID NO: 73: mature human IgG1 Fc, Cys to Ser substitution (#), allotype G1m(fa) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG
SEQ ID NO: 74: mature human IgG1 Fc, Cys to Ser substitution (#), M428L, N434S mutations
(Bold/Underlined), allotype G1m(fa) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVLHEALHSHYTQKSLSLSPG
SEQ ID NO: 75: mature human IgG1 Fc, Cys to Ser substitution (#), M428L, N434S mutations
(Bold/Underlined), allotype G1m(f) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVLHEALHSHYTQKSLSLSPG
SEQ ID NO: 76: mature human IgG1 Fc, Cys to Ser substitution (#), YTE triple mutation (bold and
underlined), allotype G1m(fa) (bold italics)
NVNHKPSNTKVDKKVEPKSS(#)DKTHTCPPCPAPELLGGPSVFLFPPKPKDTLYITREPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG
SEQ ID NO: 77: mature human IgG1 Fc, Cys to Ser substitution (#), YTE triple mutation (bold and
underlined), allotype G1m(f) (bold italics)
NVNHKPSNTKVDKKVEPKSS(#)DKTHTCPPCPAPELLGGPSVFLFPPKPKDTLYITREPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
SEQ ID NO: 78: mature human Fc IgG1, Z₁ is Cys or Ser, and wherein X₁ is Met or Tyr, X₂ is Ser or
Thr, X₃ is Thr or Glu, X₄ is Asp or Glu, and X₅ is Leu or Met, X₆ is Met or Leu, and X₇ is Asn or Ser
VNHKPSNTKVDKKVEPKSZ₁DKTHTCPPCPAPELLGGPSVFLFPPKPKDTLX₁IX₂RX₃PEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRX₄EX₅TKNQVSLTCLVKGFYPSDIAVEWESNGQPENNYKTTPPVLD
SDGSFFLYSKLTVDKSRWQQGNVFSCSVX₆HEALHX₇HYTQKSLSLSPGK
SEQ ID NO: 79: mature human Fc IgG1, Cys to Ser substitution (#), and wherein X₁ is Met or Tyr, X₂
is Ser or Thr, X₃ is Thr or Glu, X₄ is Asp or Glu, and X₅ is Leu or Met, X₆ is Met or Leu, and X₇ is Asn
or Ser
VNHKPSNTKVDKKVEPKSS(#)DKTHTCPPCPAPELLGGPSVFLFPPKPKDTLX₁IX₂RX₃PEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSRX₄EX₅TKNQVSLTCLVKGFYPSDIAVEWESNGQPENNYKTTPPVL
DSDGSFFLYSKLTVDKSRWQQGNVFSCSVX₆HEALHX₇HYTQKSLSLSPGK
SEQ ID NO: 80: mature human IgG1 Fc, Cys to Ser substitution (#), X₄ is Asp or Glu, and X₅ is Leu or
Met
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRX₄EX₅TKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 81: mature human IgG1 Fc, Cys to Ser substitution (#), allotype G1m(f) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 82: mature human IgG1 Fc, Cys to Ser substitution (#), allotype G1m(fa) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 83: mature human IgG1 Fc, Cys to Ser substitution (#), M428L, N434S mutations
(Bold/Underlined), allotype G1m(fa) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVLHEALHSHYTQKSLSLSPGK
SEQ ID NO: 84: mature human IgG1 Fc, Cys to Ser substitution (#), M428L, N434S mutations
(Bold/Underlined), allotype G1m(f) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVLHEALHSHYTQKSLSLSPGK
SEQ ID NO: 85: mature human IgG1 Fc, Cys to Ser substitution (#), YTE triple mutation (bold and
underlined), allotype G1m(fa) (bold italics)
VNHKPSNTKVDKKVEPKSS(#)DKTHTCPPCPAPELLGGPSVFLFPPKPKDTLYITREPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 86: mature human IgG1 Fc, Cys to Ser substitution (#), YTE triple mutation (bold and
underlined), allotype G1m(f) (bold italics)
VNHKPSNTKVDKKVEPKSS(#)DKTHTCPPCPAPELLGGPSVFLFPPKPKDTLYITREPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 87: mature human Fc IgG1, Z₁ is Cys or Ser, and wherein X₁ is Met or Tyr, X₂ is Ser or
Thr, X₃ is Thr or Glu, X₄ is Asp or Glu, and X₅ is Leu or Met, X₆ is Met or Leu, and X₇ is Asn or Ser
VNHKPSNTKVDKKVEPKSZ1DKTHTCPPCPAPELLGGPSVFLFPPKPKDTLX₁IX₂RX₃PEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRX₄EX₅TKNQVSLTCLVKGFYPSDIAVEWESNGQPENNYKTTPPVLD
SDGSFFLYSKLTVDKSRWQQGNVFSCSVX₆HEALHX₇HYTQKSLSLSPG
SEQ ID NO: 88: mature human Fc IgG1, Cys to Ser substitution (#), and wherein X₁ is Met or Tyr, X₂
is Ser or Thr, X₃ is Thr or Glu, X₄ is Asp or Glu, and X₅ is Leu or Met, X₆ is Met or Leu, and X₇
is Asn or Ser
VNHKPSNTKVDKKVEPKSS(#)DKTHTCPPCPAPELLGGPSVFLFPPKPKDTLX₁IX₂RX₃PEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSRX₄EX₅TKNQVSLTCLVKGFYPSDIAVEWESNGQPENNYKTTPPVL
DSDGSFFLYSKLTVDKSRWQQGNVFSCSVX₆HEALHX₇HYTQKSLSLSPG
SEQ ID NO: 89: mature human IgG1 Fc, Cys to Ser substitution (#), X₄ is Asp or Glu, and X₅ is Leu or
Met
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRX₄EX₅TKNQVSLTCLVKGFYPSDIAVEWESNGQPENNYKTTPPVLD
SDGSFFLYSKLTVDKSRWQQGNVFSCSVMHEALHNHYTQKSLSLSPG
SEQ ID NO: 90: mature human IgG1 Fc, Cys to Ser substitution (#), allotype G1m(f) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPG
SEQ ID NO: 91: mature human IgG1 Fc, Cys to Ser substitution (#), allotype G1m(fa) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
SEQ ID NO: 92: mature human IgG1 Fc, Cys to Ser substitution (#), M428L, N434S mutations
(Bold/Underlined), allotype G1m(fa) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVLHEALHSHYTQKSLSLSPG
SEQ ID NO: 93: mature human IgG1 Fc, Cys to Ser substitution (#), M428L, N434S mutations
(Bold/Underlined), allotype G1m(f) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVLHEALHSHYTQKSLSLSPG
SEQ ID NO: 94: mature human IgG1 Fc, Cys to Ser substitution (#), YTE triple mutation (bold and
underlined), allotype G1m(fa) (bold italics)
VNHKPSNTKVDKKVEPKSS(#)DKTHTCPPCPAPELLGGPSVFLFPPKPKDTLYITREPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
SEQ ID NO: 95: mature human IgG1 Fc, Cys to Ser substitution (#), YTE triple mutation (bold and
underlined), allotype G1m(f) (bold italics)
VNHKPSNTKVDKKVEPKSS(#)DKTHTCPPCPAPELLGGPSVFLFPPKPKDTLYITREPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPG
SEQ ID NO: 96: mature human Fc IgG1, J₁ is Asn or absent, J₂ is Lys or absent, Z₁ is Cys or Ser, and
wherein X₁ is Met or Tyr, X₂ is Ser or Thr, X₃ is Thr or Glu, X₄ is Asn or Ala, X₅ is Leu or Asp,
X₆ is Gln or His, X₇ is Asp or Glu, and X₈ is Leu or Met, X₉ is Met or Leu, and X₁₀ is Asn or Ser
J₁VNHKPSNTKVDKKVEPKSZ₁DKTHTCPPCPAPELLGGPSVFLFPPKPKDTLX₁IX₂RX₃PEVTCVVVD
VSHEDPEVKFNWYVDGVEVHNAKTKPREEQYX₄STYRVVSVLTVX₅HX₆DWLNGKEYKCKVSNKALP
APIEKTISKAKGQPREPQVYTLPPSRX₇EX₈TKNQVSLTCLVKGFYPSDIAVEWESNGQPENNYKTTPP
VLDSDGSFFLYSKLTVDKSRWQQGNVFSCSVX₉HEALHX₁₀HYTQKSLSLSPGJ₂
SEQ ID NO: 97: mature human Fc IgG1, Cys to Ser substitution (#), J₁ is Asn or absent, J₂ is Lys or
absent, and wherein X₄ is Asn or Ala, X₅ is Leu or Asp, X₆ is Gln or His, X₇ is Asp or Glu, and X₈ is
Leu or Met, and X₁₀ is Asn or Ser
J₁VNHKPSNTKVDKKVEPKSS(#)DKTHTCPPCPAPELLGGPSVFLFPPKPKDTLMISRTPEVTCVVVD
VSHEDPEVKFNWYVDGVEVHNAKTKPREEQYX₄STYRVVSVLTVX₅HX₆DWLNGKEYKCKVSNKALP
APIEKTISKAKGQPREPQVYTLPPSRX₇EX₈TKNQVSLTCLVKGFYPSDIAVEWESNGQPENNYKTTPP
VLDSDGSFFLYSKLTVDKSRWQQGNVFSCSVMHEALHX₁₀HYTQKSLSLSPGJ₂
SEQ ID NO: 98: mature human Fc IgG1, Cys to Ser substitution (#), DHS triple mutation (bold and
underlined), J₁ is Asn or absent, J₂ is Lys or absent, wherein X₄ is Asn or Ala, X₇ is Asp or Glu,
and X₈ is Leu or Met
J_{I}VNHKPSNTKVDKKVEPKSS(#)DKTHTCPPCPAPELLGGPSVFLFPPKPKDTLMISRTPEVTCVVVD
VSHEDPEVKFNWYVDGVEVHNAKTKPREEQYX₄STYRVVSVLTVDHHDWLNGKEYKCKVSNKALPA
PIEKTISKAKGQPREPQVYTLPPSRX₇EX₈TKNQVSLTCLVKGFYPSDIAVEWESNGQPENNYKTTPPV
LDSDGSFFLYSKLTVDKSRWQQGNVFSCSVMHEALHSHYTQKSLSLSPGJ₂
SEQ ID NO: 99: mature human Fc IgG1, Cys to Ser substitution (#), DHS triple mutation (bold and
underlined), wherein X₄ is Asn or Ala, X₇ is Asp or Glu, and X₈ is Leu or Met
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYX₄STYRVVSVLTVDHHDWLNGKEYKCKVSNKALPAP
IEKTISKAKGQPREPQVYTLPPSRX₇EX₈TKNQVSLTCLVKGFYPSDIAVEWESNGQPENNYKTTPPVL
DSDGSFFLYSKLTVDKSRWQQGNVFSCSVMHEALHSHYTQKSLSLSPGK
SEQ ID NO: 100: mature human Fc IgG1, Cys to Ser substitution (#), DHS triple mutation (bold and
underlined), wherein X₇ is Asp or Glu and X₈ is Leu or Met
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVDHHDWLNGKEYKCKVSNKALPAPI
EKTISKAKGQPREPQVYTLPPSRX₇EX₈TKNQVSLTCLVKGFYPSDIAVEWESNGQPENNYKTTPPVL
DSDGSFFLYSKLTVDKSRWQQGNVFSCSVMHEALHSHYTQKSLSLSPGK
SEQ ID NO: 101: mature human Fc IgG1, Cys to Ser substitution (#), DHS triple mutation (bold and
underlined), allotype G1m(fa) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 102: mature human Fc IgG1, Cys to Ser substitution (#), DHS triple mutation (bold and
underlined), allotype G1m(f) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVDHHDWLNGKEYKCKVSNKALPAPI
EKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 103: mature human Fc IgG1, Cys to Ser substitution (#), DHS triple mutation (bold and
underlined), allotype G1m(fa) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVDHHDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHSHYTQKSLSLSPGK
SEQ ID NO: 104: mature human Fc IgG1, Cys to Ser substitution (#), DHS triple mutation (bold and
underlined), allotype G1m(f) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVDHHDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHSHYTQKSLSLSPGK
SEQ ID NO: 105: mature human Fc IgG1, Cys to Ser substitution (#), DHS triple mutation (bold and
underlined), allotype G1m(fa) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 106: mature human Fc IgG1, Cys to Ser substitution (#), DHS triple mutation (bold and
underlined), allotype G1m(f) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVDHHDWLNGKEYKCKVSNKALPAPI
EKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 107: mature human Fc IgG1, Cys to Ser substitution (#), DHS triple mutation (bold and
underlined), allotype G1m(fa) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVDHHDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHSHYTQKSLSLSPG
SEQ ID NO: 108: mature human Fc IgG1, Cys to Ser substitution (#), DHS triple mutation (bold and
underlined), allotype G1m(f) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYNSTYRVVSVLTVDHHDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHSHYTQKSLSLSPG
SEQ ID NO: 109: mature human Fc IgG1, Cys to Ser substitution (#), Asn to Ala substitution (*), DHS
triple mutation (bold and underlined), wherein X₇ is Asp or Glu and X₈ is Leu or Met
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYA(*)STYRVVSVLTVDHHDWLNGKEYKCKVSNKALPA
PIEKTISKAKGQPREPQVYTLPPSRX₇EX₈TKNQVSLTCLVKGFYPSDIAVEWESNGQPENNYKTTPPV
LDSDGSFFLYSKLTVDKSRWQQGNVFSCSVMHEALHSHYTQKSLSLSPGK
SEQ ID NO: 110: mature human Fc IgG1, Cys to Ser substitution (#), Asn to Ala substitution (*), DHS
triple mutation (bold and underlined), allotype G1m(fa) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYA(*)STYRVVSVLTVDHHDWLNGKEYKCKVSNKALPA
PIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHSHYTQKSLSLSPGK
SEQ ID NO: 111: mature human Fc IgG1, Cys to Ser substitution (#), Asn to Ala substitution (*), DHS
triple mutation (bold and underlined), allotype G1m(f) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYA(*)STYRVVSVLTVDHHDWLNGKEYKCKVSNKALPA
PIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHSHYTQKSLSLSPGK
SEQ ID NO: 112: mature human Fc IgG1, Cys to Ser substitution (#), Asn to Ala substitution (*), DHS
triple mutation (bold and underlined), allotype G1m(fa) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYA(*)STYRVVSVLTVDHHDWLNGKEYKCKVSNKALPAP
IEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 113: mature human Fc IgG1, Cys to Ser substitution (#), Asn to Ala substitution (*), DHS
triple mutation (bold and underlined), allotype G1m(f) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYA(*)STYRVVSVLTVDHHDWLNGKEYKCKVSNKALPAP
IEKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 114: mature human Fc IgG1, Cys to Ser substitution (#), Asn to Ala substitution (*), DHS
triple mutation (bold and underlined), allotype G1m(fa) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYA(*)STYRVVSVLTVDHHDWLNGKEYKCKVSNKALPA
PIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHSHYTQKSLSLSPGK
SEQ ID NO: 115: mature human Fc IgG1, Cys to Ser substitution (#), Asn to Ala substitution (*), DHS
triple mutation (bold and underlined), allotype G1m(f) (bold italics)
NVNHKPSNTKVDKKVEPKSS(#)DKTHTCPPCPAPELLGGPSVFLFPPKPKDTLMISRTPEVTCVVVDV
SHEDPEVKFNWYVDGVEVHNAKTKPREEQYA(*)STYRVVSVLTVDHHDWLNGKEYKCKVSNKALPA
PIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHSHYTQKSLSLSPGK
SEQ ID NO: 116: mature human Fc IgG1, Cys to Ser substitution (#), Asn to Ala substitution (*), DHS
triple mutation (bold and underlined), allotype G1m(fa) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYA(*)STYRVVSVLTVDHHDWLNGKEYKCKVSNKALPAP
IEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHSHYTQKSLSLSPG
SEQ ID NO: 117: mature human Fc IgG1, Cys to Ser substitution (#), Asn to Ala substitution (*), DHS
triple mutation (bold and underlined), allotype G1m(f) (bold italics)
VNHKPSNTKVDKKVEPKSS(#)DKTHTCPPCPAPELLGGPSVFLFPPKPKDTLMISRTPEVTCVVVDVS
HEDPEVKFNWYVDGVEVHNAKTKPREEQYA(*)STYRVVSVLTVDHHDWLNGKEYKCKVSNKALPAP
IEKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHSHYTQKSLSLSPG
SEQ ID NO: 118: mature human Fc IgG1, J₁ is Asn or absent, J₂ is Lys or absent, and wherein X₄ is
Asn or Ala, X₅ is Leu or Asp, X₆ is Gln or His, X₇ is Asp or Glu, and X₈ is Leu or Met, and X₁₀ is
Asn or Ser
J₁VNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYX₄STYRVVSVLTVX₅HX₆DWLNGKEYKCKVSNKALPAPI
EKTISKAKGQPREPQVYTLPPSRX₇EX₈TKNQVSLTCLVKGFYPSDIAVEWESNGQPENNYKTTPPVL
DSDGSFFLYSKLTVDKSRWQQGNVFSCSVMHEALHX₁₀HYTQKSLSLSPGJ₂
SEQ ID NO: 119: mature human Fc IgG1, DHS triple mutation (bold and underlined), J₁ is Asn or
absent, J₂ is Lys or absent, and wherein X₄ is Asn or Ala, X₇ is Asp or Glu, and X₈ is Leu or Met
J₁VNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYX₄STYRVVSVLTVDHHDWLNGKEYKCKVSNKALPAPI
EKTISKAKGQPREPQVYTLPPSRX₇EX₈TKNQVSLTCLVKGFYPSDIAVEWESNGQPENNYKTTPPVL
DSDGSFFLYSKLTVDKSRWQQGNVFSCSVMHEALHSHYTQKSLSLSPGJ₂
SEQ ID NO: 120: mature human Fc IgG1, DHS triple mutation (bold and underlined), wherein X₄ is
Asn or Ala, and X₇ is Asp or Glu, and X₈ is Leu or Met
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYX₄STYRVVSVLTVDHHDWLNGKEYKCKVSNKALPAPI
EKTISKAKGQPREPQVYTLPPSRX₇EX₈TKNQVSLTCLVKGFYPSDIAVEWESNGQPENNYKTTPPVL
DSDGSFFLYSKLTVDKSRWQQGNVFSCSVMHEALHSHYTQKSLSLSPGK
SEQ ID NO: 121: mature human Fc IgG1, DHS triple mutation (bold and underlined), wherein X₇ is
Asp or Glu and X₈ is Leu or Met
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVDHHDWLNGKEYKCKVSNKALPAPIE
KTISKAKGQPREPQVYTLPPSRX₇EX₈TKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 122: mature human Fc IgG1, DHS triple mutation (bold and underlined), allotype
G1m(fa) (bold italics)
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVDHHDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHSHYTQKSLSLSPGK
SEQ ID NO: 123: mature human Fc IgG1, DHS triple mutation (bold and underlined), allotype G1m(f)
(bold italics)
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVDHHDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHSHYTQKSLSLSPGK
SEQ ID NO: 124: mature human Fc IgG1, DHS triple mutation (bold and underlined), allotype
G1m(fa) (bold italics)
VNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVDHHDWLNGKEYKCKVSNKALPAPIEK
TISKAKGQPREPQVYTLPPSRDELTKNQVSLTCLVKGFYPSDIAVEWESNGQPENNYKTTPPVLDSD
GSFFLYSKLTVDKSRWQQGNVFSCSVMHEALHSHYTQKSLSLSPGK
SEQ ID NO: 125: mature human Fc IgG1, DHS triple mutation (bold and underlined), allotype G1m(f)
(bold italics)
VNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVDHHDWLNGKEYKCKVSNKALPAPIEK
TISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHSHYTQKSLSLSPGK
SEQ ID NO: 126: mature human Fc IgG1, DHS triple mutation (bold and underlined), allotype
G1m(fa) (bold italics)
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVDHHDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHSHYTQKSLSLSPGK
SEQ ID NO: 127: mature human Fc IgG1, DHS triple mutation (bold and underlined), allotype G1m(f)
(bold italics)
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVDHHDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHSHYTQKSLSLSPGK
SEQ ID NO: 128: mature human Fc IgG1, DHS triple mutation (bold and underlined), allotype
G1m(fa) (bold italics)
VNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVDHHDWLNGKEYKCKVSNKALPAPIEK
TISKAKGQPREPQVYTLPPSRDELTKNQVSLTCLVKGFYPSDIAVEWESNGQPENNYKTTPPVLDSD
GSFFLYSKLTVDKSRWQQGNVFSCSVMHEALHSHYTQKSLSLSPG
SEQ ID NO: 129: mature human Fc IgG1, DHS triple mutation (bold and underlined), allotype G1m(f)
(bold italics)
VNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVDHHDWLNGKEYKCKVSNKALPAPIEK
TISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHSHYTQKSLSLSPG
SEQ ID NO: 130: mature human Fc IgG1, Asn to Ala substitution (*), DHS triple mutation (bold and
underlined), wherein X₇ is Asp or Glu and X₈ is Leu or Met
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYA(*)STYRVVSVLTVDHHDWLNGKEYKCKVSNKALPAP
IEKTISKAKGQPREPQVYTLPPSRX₇EX₈TKNQVSLTCLVKGFYPSDIAVEWESNGQPENNYKTTPPVL
DSDGSFFLYSKLTVDKSRWQQGNVFSCSVMHEALHSHYTQKSLSLSPGK
SEQ ID NO: 131: mature human Fc IgG1, Asn to Ala substitution (*), DHS triple mutation (bold and
underlined), allotype G1m(fa) (bold italics)
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYA(*)STYRVVSVLTVDHHDWLNGKEYKCKVSNKALPAP
IEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 132: mature human Fc IgG1, Asn to Ala substitution (*), DHS triple mutation (bold and
underlined), allotype G1m(f) (bold italics)
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYA(*)STYRVVSVLTVDHHDWLNGKEYKCKVSNKALPAP
IEKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 133: mature human Fc IgG1, Asn to Ala substitution (*), DHS triple mutation (bold and
underlined), allotype G1m(fa) (bold italics)
VNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYA(*)STYRVVSVLTVDHHDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHSHYTQKSLSLSPGK
SEQ ID NO: 134: mature human Fc IgG1, Asn to Ala substitution (*), DHS triple mutation (bold and
underlined), allotype G1m(f) (bold italics)
VNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYA(*)STYRVVSVLTVDHHDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHSHYTQKSLSLSPGK
SEQ ID NO: 135: mature human Fc IgG1, Asn to Ala substitution (*), DHS triple mutation (bold and
underlined), allotype G1m(fa) (bold italics)
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYA(*)STYRVVSVLTVDHHDWLNGKEYKCKVSNKALPAP
IEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 136: mature human Fc IgG1, Asn to Ala substitution (*), DHS triple mutation (bold and
underlined), allotype G1m(f) (bold italics)
NVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYA(*)STYRVVSVLTVDHHDWLNGKEYKCKVSNKALPAP
IEKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHSHYTQKSLSLSPGK
SEQ ID NO: 137: mature human Fc IgG1, Asn to Ala substitution (*), DHS triple mutation (bold and
underlined), allotype G1m(fa) (bold italics)
VNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYA(*)STYRVVSVLTVDHHDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHSHYTQKSLSLSPG
SEQ ID NO: 138: mature human Fc IgG1, Asn to Ala substitution (*), DHS triple mutation (bold and
underlined), allotype G1m(f) (bold italics)
VNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYA(*)STYRVVSVLTVDHHDWLNGKEYKCKVSNKALPAPIE
KTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHSHYTQKSLSLSPG

As defined herein, an Fc domain includes two Fc domain monomers that are dimerized by the interaction between the C_{H}3 antibody constant domains, as well as one or more disulfide bonds that form between the hinge domains of the two dimerizing Fc domain monomers. An Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present invention binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

In some embodiments, the Fc domain of the invention is an aglycosylated Fc domain (e.g., an Fc domain that maintains engagement to an Fc receptor (e.g., FcRn). For example, the Fc domain is an aglycosylated IgG1 variants that maintains engagement to an Fc receptor (e.g., an IgG1 having an amino acid substitution at N297 and/or T299 of the glycosylation motif). Exemplary aglycosylated Fc domains and methods for making aglycosylated Fc domains are known in the art, for example, as described in Sazinsky S. L. et al., Aglycosylated immunoglobulin G1 variants productively engage activating Fc receptors, PNAS, 2008, 105 (51): 20167-20172, which is incorporated herein in its entirety.

In some embodiments, the Fc domain of the invention is engineered to enhance binding to the neonatal Fc receptor (FcRn). For example, the Fc domain may include the triple mutation corresponding to M252Y/S254T/T256E (YTE) (e.g., an IgG1, such as a human or humanized IgG1 having a YTE mutation, for example SEQ ID NO: 33, SEQ ID NO: 36, SEQ ID NO: 38, SEQ ID NO: 39, SEQ ID NO: 41, SEQ ID NO: 42, SEQ ID NO: 43, SEQ ID NO: 52, SEQ ID NO: 53, SEQ ID NO: 56, or SEQ ID NO: 57). The Fc domain may include the double mutant corresponding to M428L/N434S (LS) (e.g., an IgG1, such as a human or humanized IgG1 having an LS mutation, such as SEQ ID NO: 37, SEQ ID NO: 39, SEQ ID NO: 44, SEQ ID NO: 45, SEQ ID NO: 46, SEQ ID NO: 50, SEQ ID NO: 51, SEQ ID NO: 54, SEQ ID NO: 55, or SEQ ID NO: 59). The Fc domain may include the single mutant corresponding to N434H (e.g., an IgG1, such as a human or humanized IgG1 having an N434H mutation). The Fc domain may include the single mutant corresponding to C220S (e.g., an IgG1, such as a human or humanized IgG1 having a C220S mutation, such as SEQ ID NO: 34, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, and SEQ ID NO: 95). The Fc domain may include a quadruple mutant corresponding to C220S/L309D/Q311H/N434S (CDHS) (e.g., an IgG1, such as a human or humanized IgG1 having a DHS mutation, such as SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, and SEQ ID NO: 117). The Fc domain may include a triple mutant corresponding to L309D/Q311H/N434S (DHS) (e.g., an IgG1, such as a human or humanized IgG1 having a DHS mutation, such as SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, and SEQ ID NO: 138). The Fc domain may include a combination of one or more of the above-described mutations that enhance binding to the FcRn. Enhanced binding to the FcRn may increase the half-life Fc domain-containing conjugate. For example, incorporation of one or more amino acid mutations that increase binding to the FcRn (e.g., a YTE mutation, an LS mutation, or an N434H mutation) may increase the half-life of the conjugate by 5%, 10%, 15%, 20%, 30%, 40%, 50%, 60%, 70%, 80%, 90%, 100%, 200%, 300%, 400%, 500% or more relative to a conjugate having the corresponding Fc domain without the mutation that enhances FcRn binding. Exemplary Fc domains with enhanced binding to the FcRN and methods for making Fc domains having enhanced binding to the FcRN are known in the art, for example, as described in Maeda, A. et al., Identification of human IgG1 variant with enhanced FcRn binding and without increased binding to rheumatoid factor autoantibody, MABS, 2017, 9 (5): 844-853, which is incorporated herein in its entirety.

As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-138 may be mutated to include a YTE mutation, an LS mutation, and/or an N434H mutation by mutating the “corresponding residues” of the amino acid sequence.

As used herein, a sulfur atom “corresponding to” a particular cysteine residue of a particular SEQ ID NO. should be understood to include the sulfur atom of any cysteine residue that one of skill in the art would understand to align to the particular cysteine of the particular sequence. The protein sequence alignment of human IgG1 (UniProtKB: P01857; SEQ ID NO: 142), human IgG2 (UniProtKB: P01859; SEQ ID NO: 143), human IgG3 (UniProtKB: P01860; SEQ ID NO: 144), and human IgG4 (UniProtKB: P01861; SEQ ID NO: 145) is provided below (aligned with Clustal Omega Multiple Pairwise Alignment). The alignment indicates cysteine residues (e.g., sulfur atoms of cysteine residues) that “correspond to” one another (in boxes and indicated by the ⋅ symbol). One of skill in the art would readily be able to perform such an alignment with any IgG variant of the invention to determine the sulfur atom of a cysteine that corresponds to any sulfur atom of a particular cysteine of a particular SEQ ID NO. described herein (e.g., any one of SEQ ID NOs: 1-138). For example, one of skill in the art would readily be able to determine that Cys10 of SEQ ID NO: 10 (the first cysteine of the conserved CPPC motif of the hinge region of the Fc domain) corresponds to, for example, Cys109 of IgG1, Cys106 of IgG2, Cys156 of IgG3, Cys29 of SEQ ID NO: 1, Cys9 of SEQ ID NO: 2, Cys30 of SEQ ID NO: 3, or Cys10 of SEQ ID NO: 10.

In some embodiments, the Fc domain of the invention has the sequence of any one of SEQ ID NOs: 39-138 may further include additional amino acids at the N-terminus (Xaa)x and/or additional amino acids at the C-terminus (Xaa)z, wherein Xaa is any amino acid and x and z are a whole number greater than or equal to zero, generally less than 100, preferably less than 10 and more preferably 0, 1, 2, 3, 4, or 5. In some embodiments, the additional amino acids are least 70% (e.g., 70%, 75%, 80%, 85%, 90%, 95%, 96%, 97%, 98%, 99%, or 100%) identical to one or more consecutive amino acids of SEQ ID NO: 81. For example, the additional amino acids may be a single amino acid on the C-terminus corresponding to Lys330 of IgG1 (SEQ ID NO: 119).

As used herein, a nitrogen atom “corresponding to” a particular lysine residue of a particular SEQ ID NO. should be understood to include the nitrogen atom of any lysine residue that one of skill in the art would understand to align to the particular lysine of the particular sequence. The protein sequence alignment of human IgG1 (UniProtKB: P01857; SEQ ID NO: 142), human IgG2 (UniProtKB: P01859; SEQ ID NO: 143), human IgG3 (UniProtKB: P01860; SEQ ID NO: 144), and human IgG4 (UniProtKB: P01861; SEQ ID NO: 145) is provided below (aligned with Clustal Omega Multiple Pairwise Alignment). The alignment indicates lysine residues (e.g., nitrogen atoms of lysine residues) that “correspond to” one another (in boxes and indicated by the * symbol). One of skill in the art would readily be able to perform such an alignment with any IgG variant of the invention to determine the nitrogen atom of a lysine that corresponds to any nitrogen atom of a particular lysine of a particular SEQ ID NO. described herein (e.g., any one of SEQ ID NOs: 1-138). For example, one of skill in the art would readily be able to determine that Lys35 of SEQ ID NO: 10 corresponds to, for example, Lys129 of IgG1, Lys126 of IgG2, Lys176 of IgG3, Lys51 of SEQ ID NO: 1, Lys31 of SEQ ID NO: 2, Lys50 of SEQ ID NO: 3, or Lys30 of SEQ ID NO: 10.

Protein sequence alignment of
IgG1 (SEQ ID NO: 142), IgG2 (SEQ ID NO: 143),
IgG3 (SEQ IDNO: 144), and IgG4 (SEQ ID NO: 145)

| | |
|---|---|
| human_IgG1 human_IgG2 human_IgG3 | 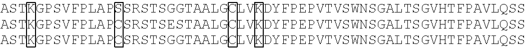 |
| human_IgG4 | ASTKGPSVFPLAPCSRSTSESTAALGCLVKDYFFEPVTVSWNSGALTSGVHTFPAVLASS |
| | * * * * |
| human_IgG1 human_IgG2 human_IgG3 | 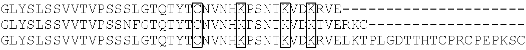 |
| human_IgG4 | GLYSLSSVVTVPSSSLGTKTYTCNVDHKPSNIKVDKRVESK------------------- |
| | * * * * |
| human_IgG1 human_IgG2 human_IgG3 human_IgG4 | 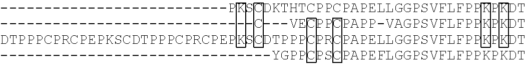 |
| | * * * * * * |
| human_IgG1 human_IgG2 human_IgG3 | 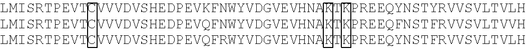 |
| human_IgG4 | LMISRTPEVTCVVVDVSQEDPEVQFRWYVDGVEVHNAKTKPREEQFNSTYRVVSVLTVLH |
| | * * * |
| human_IgG1 human_IgG2 human_IgG3 | 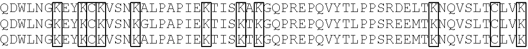 |
| human_IgG4 | QDWLNGKEYKCKVSNKGLPGGIEKTISKGKGQPREPQVYTLPPSQEEMTKNQVSLTCLVK |
| | * *** * * * * * * * |
| human_IgG1 human_IgG2 human_IgG3 | 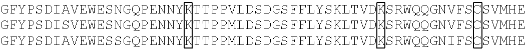 |
| human_IgG4 | GFYPSDIAVEWESNGQPENNYKTTPPVLDSDGSFFLYSRLTVDKSRWQEGNIFSCSVMHE |
| | * * * |
| human_IgG1 human_IgG2 human_IgG3 | 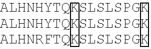 |
| human_IgG4 | ALHNNYTQKSLSLSPGK |
| | * * |

### Pharmaceutical Compositions and Preparations

A conjugate described herein may be formulated in a pharmaceutical composition for use in the methods described herein. In some embodiments, a conjugate described herein may be formulated in a pharmaceutical composition alone. In some embodiments, a conjugate described herein may be formulated in combination with an antiviral agent or antiviral vaccine in a pharmaceutical composition. In some embodiments, the pharmaceutical composition includes a conjugate of Formula (I) and pharmaceutically acceptable carriers and excipients.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acid residues such as glycine, glutamine, histidine, and lysine, and carbohydrates such as glucose, mannose, sucrose, and sorbitol.

Examples of other excipients include, but are not limited to, antiadherents, binders, coatings, compression aids, disintegrants, dyes, emollients, emulsifiers, fillers (diluents), film formers or coatings, flavors, fragrances, glidants (flow enhancers), lubricants, sorbents, suspensing or dispersing agents, or sweeteners. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The pharmaceutical composition may be formed in a unit dose form as needed. The amount of active component, e.g., a conjugate of Formula (I) included in the unit dose form are such that a dose within the range of 10-900 mg (e.g., from 10 mg to 20 mg, from 15 mg to 30 mg, from 10 mg to 25 mg, from 15 mg to 25 mg, from 20 mg to 30 mg, from 25 mg to 35 mg, from 25 mg to 35 mg, from 30 mg to 40 mg, from 35 mg to 50 mg, from 30 mg to 45 mg, from 40 mg to 50 mg, from 45 mg to 50 mg, from 50 mg to 75 mg, from 50 mg to 100 mg, from 50 mg to 125 mg, from 50 mg to 150 mg, from 50 mg to 175 mg, from 50 mg to 200 mg, from 50 mg to 225 mg, from 50 mg to 250 mg, from 60 mg to 75 mg, from 60 mg to 100 mg, from 60 mg to 125 mg, from 60 mg to 150 mg, from 60 mg to 175 mg, from 60 mg to 200 mg, from 60 mg to 225 mg, from 60 mg to 250 mg, from 70 mg to 75 mg, from 70 mg to 100 mg, from 70 mg to 125 mg, from 70 mg to 150 mg, from 70 mg to 175 mg, from 70 mg to 200 mg, from 70 mg to 225 mg, from 70 mg to 250 mg, from 80 mg to 100 mg, from 80 mg to 125 mg, from 80 mg to 150 mg, from 80 mg to 175 mg, from 80 mg to 200 mg, from 80 mg to 225 mg, from 80 mg to 250 mg, from 90 mg to 100 mg, from 90 mg to 125 mg, from 90 mg to 150 mg, from 90 mg to 175 mg, from 90 mg to 200 mg, from 90 mg to 225 mg, from 90 mg to 250 mg, from 100 mg to 125 mg, from 100 mg to 150 mg, from 100 mg to 175 mg, from 100 mg to 200 mg, from 100 mg to 225 mg, from 100 mg to 250 mg, from 150 mg to 175 mg, from 150 mg to 200 mg, from 150 mg to 225 mg, from 150 mg to 250 mg, from 200 mg to 225 mg, from 200 mg to 250 mg, from 200 mg to 275 mg, from 200 mg to 300 mg, from 200 mg to 325 mg, and from 200 mg to 350 mg, 300 mg to 325 mg, from 300 mg to 350 mg, from 300 mg to 375 mg, from 300 mg to 400 mg, from 300 mg to 425 mg, from 300 mg to 450 mg, from 400 mg to 425 mg, from 400 mg to 450 mg, from 400 mg to 475 mg, from 400 mg to 500 mg, from 400 mg to 525 mg, from 400 mg to 550 mg, from 500 mg to 525 mg, from 500 mg to 550 mg, from 500 mg to 575 mg, from 500 mg to 600 mg, from 500 mg to 625 mg, from 500 mg to 650 mg, from 600 mg to 625 mg, from 600 mg to 650 mg, from 600 mg to 675 mg, from 600 mg to 700 mg, from 600 mg to 725 mg, from 600 mg to 750 mg, from 700 mg to 725 mg, from 700 mg to 750 mg, from 700 mg to 775 mg, from 700 mg to 800 mg, from 700 mg to 825 mg, from 700 mg to 850 mg, from 800 mg to 825 mg, from 800 mg to 850 mg, from 800 mg to 875 mg, and from 800 mg to 900 mg) is provided. In some embodiments, the unit dosage form includes 150 mg of a conjugate of Formula (I) (e.g., Conjugate A). In some embodiments, the unit dosage form includes 300 mg of a conjugate of Formula (I) (e.g., Conjugate A). In some embodiments, the unit dosage form includes 900 mg of a conjugate of Formula (I) (e.g., Conjugate A).

### Routes of Administration and Dosages

In any of the methods described herein, conjugates herein may be administered by any appropriate route for treating or protecting against an influenza infection, or for preventing, stabilizing, or inhibiting the proliferation or spread of an influenza virus. In some embodiments, administering comprises administration of any of a conjugate of Formula (I) intramuscularly, intravenously (e.g., as a sterile solution and in a solvent system suitable for intravenous use), or subcutaneously.

The dosage of a conjugate described herein is between 10 and 900 mg (e.g., from 10 mg to 20 mg, from 15 mg to 30 mg, from 10 mg to 25 mg, from 15 mg to 25 mg, from 20 mg to 30 mg, from 25 mg to 35 mg, from 25 mg to 35 mg, from 30 mg to 40 mg, from 35 mg to 50 mg, from 30 mg to 45 mg, from 40 mg to 50 mg, from 45 mg to 50 mg, from 50 mg to 75 mg, from 50 mg to 100 mg, from 50 mg to 125 mg, from 50 mg to 150 mg, from 50 mg to 175 mg, from 50 mg to 200 mg, from 50 mg to 225 mg, from 50 mg to 250 mg, from 60 mg to 75 mg, from 60 mg to 100 mg, from 60 mg to 125 mg, from 60 mg to 150 mg, from 60 mg to 175 mg, from 60 mg to 200 mg, from 60 mg to 225 mg, from 60 mg to 250 mg, from 70 mg to 75 mg, from 70 mg to 100 mg, from 70 mg to 125 mg, from 70 mg to 150 mg, from 70 mg to 175 mg, from 70 mg to 200 mg, from 70 mg to 225 mg, from 70 mg to 250 mg, from 80 mg to 100 mg, from 80 mg to 125 mg, from 80 mg to 150 mg, from 80 mg to 175 mg, from 80 mg to 200 mg, from 80 mg to 225 mg, from 80 mg to 250 mg, from 90 mg to 100 mg, from 90 mg to 125 mg, from 90 mg to 150 mg, from 90 mg to 175 mg, from 90 mg to 200 mg, from 90 mg to 225 mg, from 90 mg to 250 mg, from 100 mg to 125 mg, from 100 mg to 150 mg, from 100 mg to 175 mg, from 100 mg to 200 mg, from 100 mg to 225 mg, from 100 mg to 250 mg, from 150 mg to 175 mg, from 150 mg to 200 mg, from 150 mg to 225 mg, from 150 mg to 250 mg, from 200 mg to 225 mg, from 200 mg to 250 mg, from 200 mg to 275 mg, from 200 mg to 300 mg, from 200 mg to 325 mg, and from 200 mg to 350 mg, 300 mg to 325 mg, from 300 mg to 350 mg, from 300 mg to 375 mg, from 300 mg to 400 mg, from 300 mg to 425 mg, from 300 mg to 450 mg, from 400 mg to 425 mg, from 400 mg to 450 mg, from 400 mg to 475 mg, from 400 mg to 500 mg, from 400 mg to 525 mg, from 400 mg to 550 mg, from 500 mg to 525 mg, from 500 mg to 550 mg, from 500 mg to 575 mg, from 500 mg to 600 mg, from 500 mg to 625 mg, from 500 mg to 650 mg, from 600 mg to 625 mg, from 600 mg to 650 mg, from 600 mg to 675 mg, from 600 mg to 700 mg, from 600 mg to 725 mg, from 600 mg to 750 mg, from 700 mg to 725 mg, from 700 mg to 750 mg, from 700 mg to 775 mg, from 700 mg to 800 mg, from 700 mg to 825 mg, from 700 mg to 850 mg, from 800 mg to 825 mg, from 800 mg to 850 mg, from 800 mg to 875 mg, and from 800 mg to 900 mg). In some embodiments, the dosage administered to the human subject is 150 mg of a conjugate of Formula (I) (e.g., Conjugate A). In some embodiments, the dosage administered to the human subject is 300 mg of a conjugate of Formula (I) (e.g., Conjugate A). In some embodiments, the dosage administered to the human subject is 900 mg of a conjugate of Formula (I) (e.g., Conjugate A). A conjugate of Formula (I) or a pharmaceutical composition thereof may be administered to a human subject one time, e.g., prior to flu season.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1: Clinical Trial Protocol

We performed a proof-of-concept, randomized, double-blind, placebo-controlled, Phase 2a study to assess the prophylactic antiviral activity against influenza H3N2 A/Perth/16/2009, safety, tolerability, and pharmacokinetics of Conjugate A via a human viral challenge model. The Conjugate A used in this study has a DAR of 4.5.

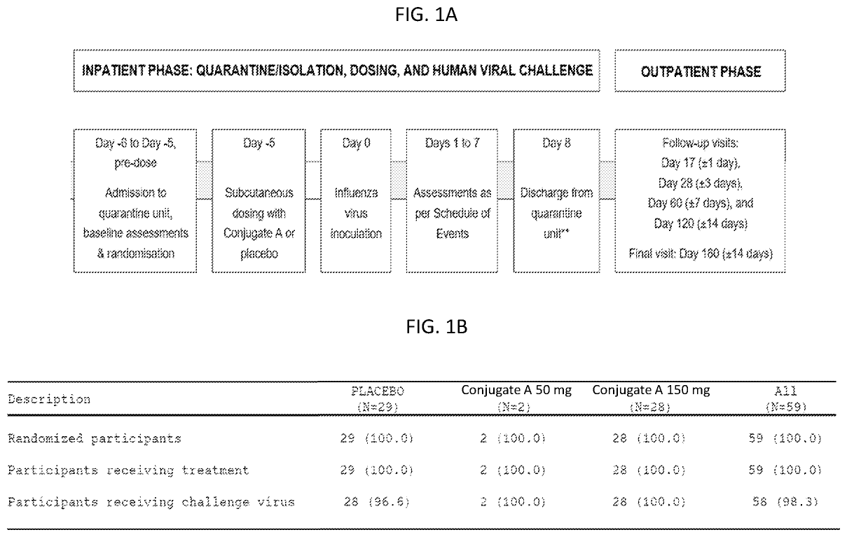

To evaluate the prophylactic efficacy of Conjugate A in terms of reduction of area under the viral load-time curve (VL-AUC) after influenza viral challenge when compared to placebo, we used quantitative reverse transcriptase-polymerase chain reaction (qRT-PCR) on nasal samples starting a day post viral challenge (Day 1, pm) up to Day 8 (am) to determine the area under the viral load-time curve (VL AUC) of influenza challenge virus (Table 1). The study protocol is summarized in FIG. 1A.

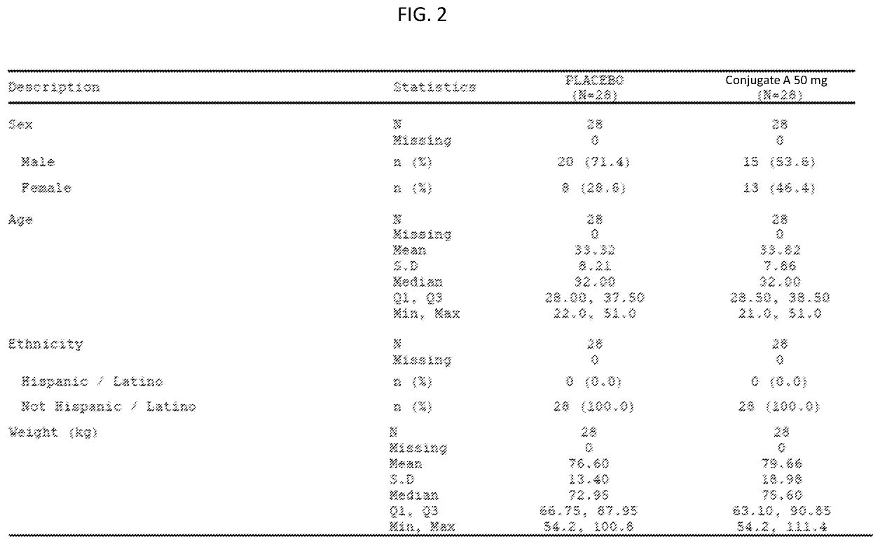

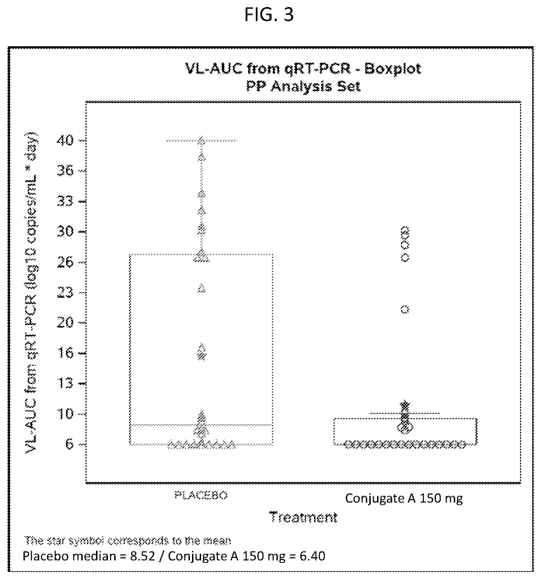

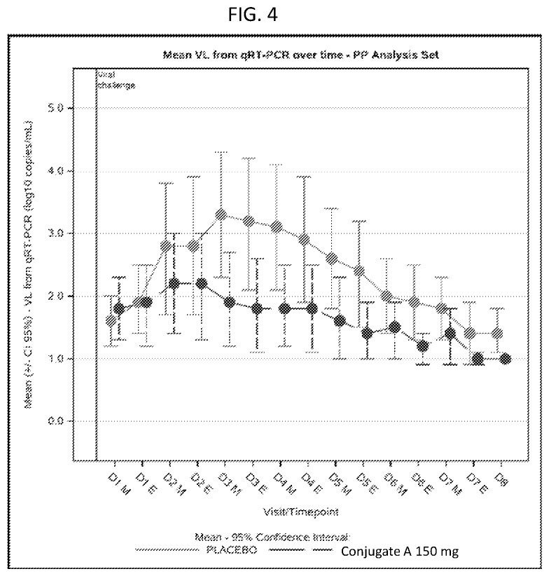

A summary of the disposition of the trial is shown in FIG. 1B. The demographics of human volunteers is shown in FIG. 2. A boxplot of the VL-AUC data in Table 1 is shown in FIG. 3. The mean viral load from qRT-PCR over eight days is shown in FIG. 4.

TABLE 1
Summary of Area Under the Viral Load-
time Curve (VL-AUC) from qRT-PCR.

| | Placebo | Conjugate A 150 mg |
|---|---|---|
| N | 28 | 28 |
| Mean | 16.09 | 10.70 |
| SD | 11.86 | 8.01 |
| Median | 8.52 | 6.40 |
| Q1, Q3 | 6.39, 27.30 | 6.38, 9.23 |
| 95% CI of the Mean | [11.49; 20.69] | [7.60; 13.81] |

Conjugate A versus placebo comparison

| | |
|---|---|
| Hodges-Lehmann | −1.2 |
| estimation of the | |
| location shift | |
| 95% CI | [−5.7; 0.0] |
| One sided p-value | 0.0390 |
| from Wilcoxon | |
| rank sum test | |

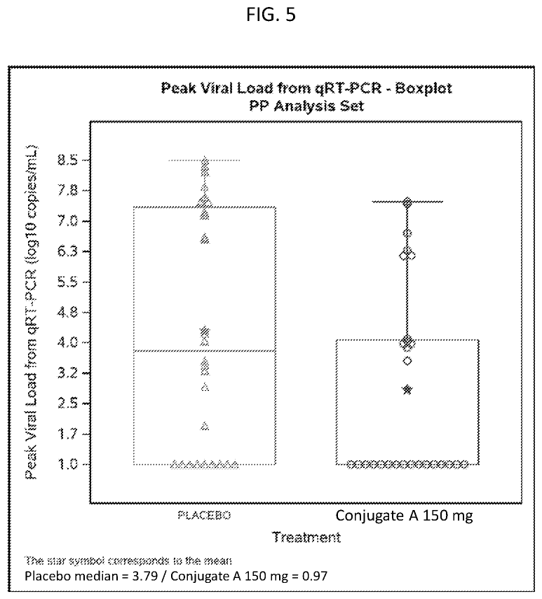

To evaluate the effect of Conjugate A in reducing or shortening viral replication after influenza viral challenge compared to placebo, we used quantifiable qRT-PCR measurements in nasal samples from Day 1 (pm) up to Day 8 (am) to determine peak viral load of influenza as defined by the maximum viral load (Table 2). A boxplot plot of the peak viral load from qRT-PCR in Table 2 is shown in FIG. 5.

TABLE 2
Summary of Peak Viral Load from qRT-PCR.

| | Placebo | Conjugate A 150 mg |
|---|---|---|
| N | 28 | 28 |
| Mean | 4.28 | 2.84 |
| SD | 2.94 | 2.40 |
| Median | 3.79 | 0.97 |
| Q1, Q3 | 0.97, 7.38 | 0.97, 4.07 |
| 95% CI of the Mean | [3.14; 5.42] | [1.90; 3.77] |

Conjugate A versus placebo comparison

| | |
|---|---|
| Hodges-Lehmann | −1.4 |
| estimation of the | |
| location shift | |
| 95% CI | [−2.9; −0.0] |
| One sided p-value | 0.0185 |
| from Wilcoxon | |
| rank sum test | |

To evaluate the effect of Conjugate A in reducing the incidence of influenza infection due to an influenza viral challenge, compared to placebo, we used qRT-PCR to detect number of incidences of influenza infections, summarized in Table 3. qRT-PCR-confirmed influenza infection, defined as 2 quantifiable (≥lower limit of quantification [LLOQ]) qRT-PCR measurements (reported on 2 or more independent samples over 2 days), from Day 1 (pm) up to Day 8 (am). Occurrence of at least 1 positive quantitative (≥LLOQ) cell culture measurement in nasal samples, from Day 1 (pm) up to Day 8 (am). qRT-PCR-confirmed symptomatic influenza infection, defined as: qRT-PCR-confirmed influenza infection (2 quantifiable [≥LLOQ] qRT-PCR measurements [reported on 2 or more independent samples over 2 days]), from Day 1 (pm) up to Day 8 (am), AND Symptoms ≥2 at a single time point. qRT-PCR-confirmed moderately severe symptomatic influenza infection, defined as: qRT-PCR-confirmed influenza infection (2 quantifiable [≥LLOQ] qRT-PCR measurements [reported on 2 or more independent samples over 2 days]), from Day 1 (pm) up to Day 8 (am), AND Any symptoms of grade ≥2 at a single time point. Culture lab-confirmed symptomatic influenza infection, defined as: Lab-confirmed culturable influenza infection (1 quantifiable [≥LLOQ] cell culture measurement), from Day 1 (pm) up to Day 8 (am), AND Symptoms ≥2 at a single time point.

TABLE 3
Summary of influenza infection incidences.

| | | |
|---|---|---|
| Placebo | Conjugate | |
| N = 28 | A N = 28 | p-value * |

qRT-PCR Confirmed Influenza Infection

| | | |
|---|---|---|
| 14 (50%) | 6 (21.4%) | 0.0248 |

qRT-PCR Confirmed Symptomatic Influenza Infection

| | | |
|---|---|---|
| 9 (32.1%) | 4 (14%) | 0.1023 |

qRT-PCR-Confirmed Moderately to
Severe Symptomatic Influenza Infection

| | | |
|---|---|---|
| 7 (25.0%) | 3 (10.7%) | 0.1477 |

* Fisher Exact test, one-sided

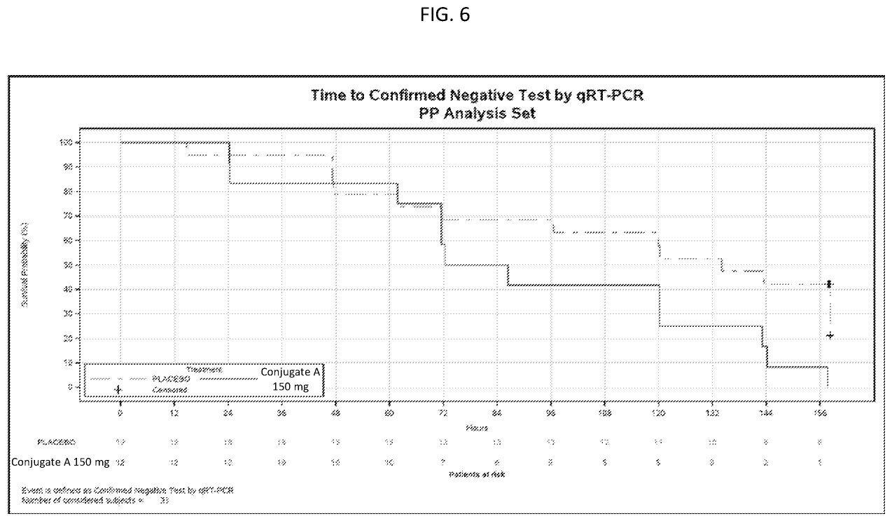

To evaluate the effect of Conjugate A in reducing or shortening viral shedding after influenza viral challenge compared to placebo, we measured the time (hours) to confirmed negative test using quantifiable qRT-PCR measurements in nasal samples from Day 1 (pm) to first confirmed undetectable assessment after peak measure. The results are summarized in Table 4 and FIG. 6.

TABLE 4
Summary of time to confirmed negative test by qRT-PCR.

| | | Placebo | Conjugate A 150 mg |
|---|---|---|---|
| N | N | 28 | 28 |
| Included | n (%) | 19 (67.9%) | 12 (42.9%) |
| Excluded * | n (%) | 9 (32.1%) | 16 (57.1%) |
| With Event | n (%) | 12 (63.2%) | 12 (100%) |
| Censored | n (%) | 7 (36.8%) | 0 |
| Kaplan-Meier | Median | 134.1 | 79.4 |
| Estimates (hours) | 95% CI | [61.9; NC] | [24.1; 143.1] |

| | Conjugate A versus placebo comparison | |
|---|---|---|
| 1-sided p-value from | | 0.0613 |
| Gehan-Wilcoxon test | | |

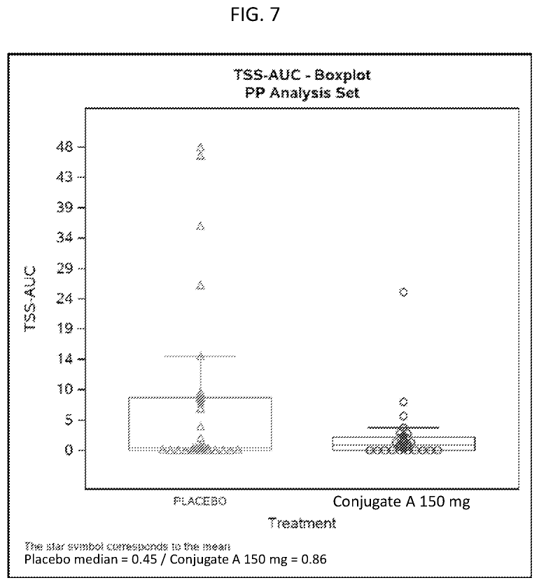

To evaluate the effect of Conjugate A in reducing clinical symptoms due to influenza viral challenge compared to placebo, we measured the Area under the curve over time of total clinical symptoms score (TSS-AUC) using the graded symptom scoring system, collected 3 times daily from Day 1 (am) up to Day 8 (am). The TSS-AUC data is summarized in Table 5 and in FIG. 7.

TABLE 5
Summary of Area Under the Curve over time
of Total Clinical Symptoms Score (TSS-AUC).

| | Placebo | Conjugate A 150 mg |
|---|---|---|
| N | 28 | 28 |
| Mean | 7.66 | 2.22 |
| SD | 14.13 | 4.86 |
| Median | 0.45 | 0.86 |
| Q1, Q3 | 0.00, 8.40 | 0.00, 2.15 |
| 95% CI of the Mean | [2.18; 13.14] | [0.33; 4.10] |

Conjugate A versus placebo comparison

| | |
|---|---|
| Hodges-Lehmann | −5.4 |
| estimation of the | |
| location shift | |
| 95% CI | [−11.2; 0.2] |
| One sided p-value | 0.2877 |
| from Wilcoxon | |
| rank sum test | |

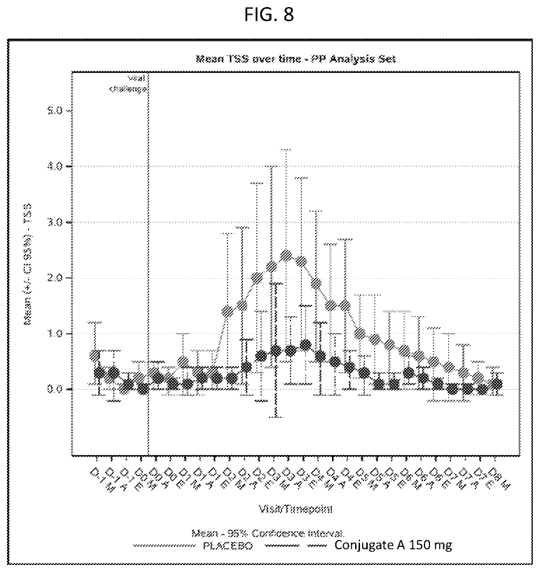

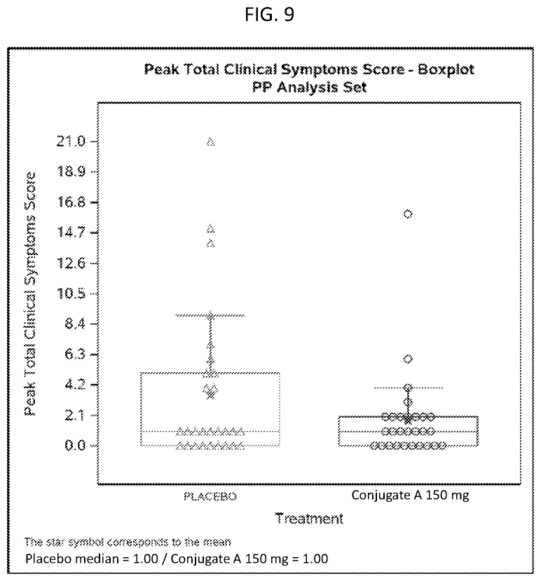

To evaluate the effect of Conjugate A in reducing clinical symptoms due to influenza viral challenge compared to placebo, we measured the Total Clinical Symptoms Score (TSS). The peak symptoms diary card score consists of the peak of TSS as measured by graded symptom scoring system collected 3 times daily from Day 1 (am) up to Day 8 (am). Peak daily symptom score is the individual maximum daily sum of symptom score from Day 1 up to Day 8. The TSS results are summarized in Table 6, and the mean TSS over time is shown in FIG. 8. A boxplot of the peak TSS data is shown in FIG. 9.

TABLE 6
Summary of peak Total Clinical Symptoms Score (TSS).

| | Placebo | Conjugate A 150 mg |
|---|---|---|
| N | 28 | 28 |
| Mean | 3.54 | 1.79 |
| SD | 5.34 | 3.12 |
| Median | 1.00 | 1.00 |
| Q1, Q3 | 0.00, 5.00 | 0.00, 2.00 |
| 95% CI of the Mean | [1.47; 5.61] | [0.58; 3.00] |

Conjugate A versus placebo comparison

| | |
|---|---|
| Hodges-Lehmann | −1.8 |
| estimation of the | |
| location shift | |
| 95% CI | [−4.1; 0.6] |
| One sided p-value | 0.2517 |
| from Wilcoxon | |
| rank sum test | |

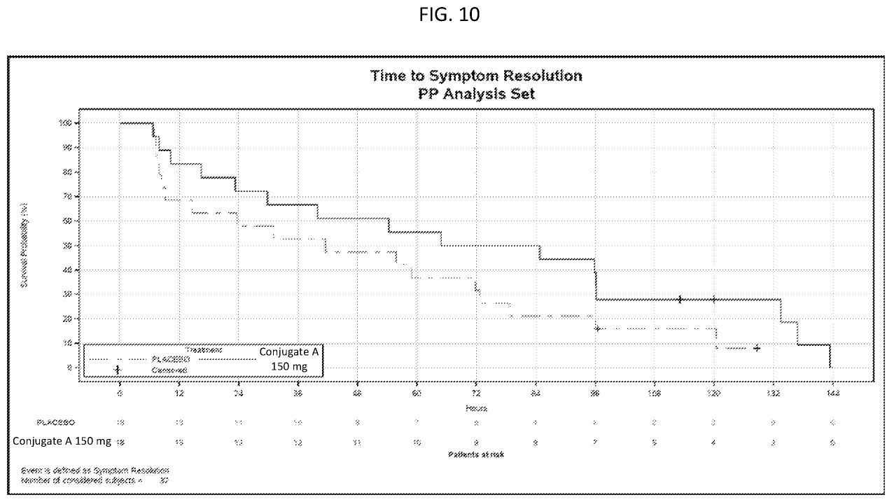

To further evaluate the effect of Conjugate A, in reducing clinical symptoms due to influenza viral challenge compared to placebo, we measured the time to symptom resolution by a graded daily symptom score system from time of peak daily symptom score to time of returning to baseline score. The time to symptom resolution data is summarized in Table 7 and in FIG. 10.

TABLE 7
Summary of time to symptom resolution.

| | Placebo | Conjugate A 150 mg |
|---|---|---|
| N | 28 | 28 |
| Mean | 3.54 | 1.79 |
| SD | 5.34 | 3.12 |
| Median | 1.00 | 1.00 |
| Q1, Q3 | 0.00, 5.00 | 0.00, 2.00 |
| 95% CI of the Mean | [1.47; 5.61] | [0.58; 3.00] |

Conjugate A versus placebo comparison

| | |
|---|---|
| Hodges-Lehmann | −1.8 |
| estimation of the | |
| location shift | |
| 95% CI | [−4.1; 0.6] |
| One sided p-value | 0.2517 |
| from Wilcoxon | |
| rank sum test | |

### Study Rationale

This is a study to determine the prophylactic antiviral activity of a single subcutaneous administration of Conjugate A against experimental influenza infection and to confirm its safety, tolerability, and PK in a healthy adult population inoculated with the influenza H3N2 A/Perth/16/2009 challenge strain. Due to the current epidemiological situation with very low influenza incidence worldwide, it is challenging to demonstrate proof of concept of therapeutic or prophylactic efficacy of anti-influenza compounds in a natural infection setting. The HVC model offers a setting that is independent of the number of influenza cases at a given time. Also, this model has a track record of safety and has demonstrated proof-of-concept efficacy for numerous antiviral compounds and vaccines that were later found to be also efficacious in larger clinical studies in the setting of natural infection. It is important to establish proof of concept and minimally effective concentrations in this study in order to better design studies to establish the safety and efficacy of Conjugate A.

Influenza challenge strains have been used for over 20 years and have helped assess numerous antiviral, immunomodulating, and vaccine therapies.

### Challenge Agent

The challenge agent used in this study is influenza H3N2 A/Perth/16/2009. The challenge agent stock was manufactured under current GMP (CGMP). The challenge agent stock has undergone quality testing performed during manufacturing (identity, appearance, sterility, infectivity, and contaminants) according to pre-determined specifications, and has subsequently also passed an extensive panel of adventitious agent testing. The challenge agent is stored in a secure −80° C. freezer (normal temperature range −60° C. to −90° C.).

### Example 2: Single Dose Pharmacokinetics of Conjugate A in Humans

This is a first-in-human, Phase 1, single-center, prospective, randomized, double-blind study of ascending single doses of Conjugate A administered intramuscularly (IM) or subcutaneously (SQ) to healthy adult subjects followed by another single dose of Conjugate A administered by the same route three months or five effective half-lives, whichever is longer, after the first dose in the middle and high dose groups (dosing may be adjusted for tolerability). The Conjugate A used in this study has a DAR of 4.5. Plasma Conjugate A concentrations were determined for subjects randomized to receive Conjugate A using a validated hybrid immunoassay LC-MS method with lower limit of quantitation (LLOQ) of 0.1 μg/mL. The goal is to assess the plasma pharmacokinetics of Conjugate A in humans.

Dose levels of Conjugate A followed an ascending single dose design with the starting dose based on findings from 3-month rat and monkey toxicology studies. Within each route (IM and SQ dose, route assignment is unblinded), subjects were randomized to receive a single dose of Conjugate A Injection or saline placebo (treatment assignment is blinded) according to the design in Table 8.

TABLE 8
Pharmacokinetics study design in humans.

| | Number of Subjects (N = 77) | | | | | |
|---|---|---|---|---|---|---|
| | Intramuscular (IM) | | | Subcutaneous (SQ) | | |
| Dose | | Conjugate A | Placebo | | Conjugate A | Placebo |
| (mg)^{a} | Cohort | (n = 24) | (n = 9) | Cohort | (n = 32) | (n = 12) |
| 50 | Cohort 1A (sentinel) | 1 | 1 | Cohort 1B | 1 | 1 |
| | | | | (sentinel) | | |
| 50 | Cohort 1A (main) | 7 | 2 | Cohort 1B (main) | 7 | 2 |
| 150 | Cohort 2A (sentinel) | 1 | 1 | Cohort 2B | 1 | 1 |
| | | | | (sentinel) | | |
| 150 | Cohort 2A (main) | 7 | 2 | Cohort 2B (main) | 7 | 2 |
| 450 | Cohort 3A (sentinel) | 1 | 1 | Cohort 3B | 1 | 1 |
| | | | | (sentinel) | | |
| 450 | Cohort 3A (main) | 7 | 2 | Cohort 3B (main) | 7 | 2 |
| 900 | NA | NA | NA | Cohort 4B | 1 | 1 |
| | | | | (sentinel) | | |
| 900 | NA | NA | NA | Cohort 4B (main) | 7 | 2 |

NA = not applicable
^{a}Note:
Immediately prior to the second single dose, within each dose and route, any dropouts in the sentinel groups were randomly replaced with subjects from the main groups, retaining their original treatment assignment (i.e., subjects assigned to placebo remained placebo, and subjects assigned to Conjugate A remained Conjugate A).

Each cohort was divided into two groups identified as “sentinel” groups (randomized 1:1) and “main” groups (randomized 7:2):
Low dose (50 mg) level: Cohort 1A (IM) and Cohort 1B (SQ)—each route n=2 sentinel and n=9 main
Mid dose (150 mg) level: Cohort 2A (IM) and Cohort 2B (SQ)—each route n=2 sentinel and n=9 main.
High dose (450 mg) level: Cohort 3A (IM) and Cohort 3B (SQ)—each route n=2 sentinel and n=9 main
Highest dose (900 mg) level: Cohort 4B (SQ)—n=2 sentinel and n=9 main

For each route, the sentinel or first group of two subjects (1 Conjugate A: 1 placebo) at a dose level was administered blinded study drug and closely monitored for safety for at least one week. The Principal Investigator (PI) and Sponsor reviewed blinded safety data after the initial sentinel group subjects at a dose level have completed dosing and followed for a minimum of one week. No drug-related serious adverse events have occurred, so the subjects in the corresponding main group were dosed one week after the sentinel group was dosed. When the dose/route of administration was determined to be safe and well tolerated ≥14 days after dosing, the next cohort of subjects were enrolled and randomized to receive the next higher dose level of Conjugate A Injection or placebo.

Subjects in Cohort 4B received a single dose of Conjugate A Injection or placebo (SQ). Subjects in Cohorts 2A and 2B and Cohorts 3A and 3B received a second single dose of Conjugate A or placebo after washout of five effective half-lives after the first dose if it is determined that the safety and tolerability of the first dose was acceptable upon review of the cumulative safety data. Immediately prior to the second single dose, within each dose and route, any dropouts in the sentinel groups were randomly replaced with subjects from the main groups, retaining their original treatment assignment (i.e., subjects assigned to placebo will remain placebo, and subjects assigned to Conjugate A will remain Conjugate A; this may require unblinded personnel to oversee). The PI and Sponsor reviewed the blinded safety data after the initial sentinel group subjects at a dose level completed dosing and followed for the minimum observation period as determined during the first single dose, which was seven days. No drug-related serious adverse events occurred, so the subjects in the corresponding main group were dosed one week after the sentinel group was dosed.

Pharmacokinetics (PK) were determined by analyzing plasma samples for concentration of Conjugate A obtained from subjects who receive Conjugate A Injection in each cohort at various time points after administration of the first and second single doses of the study drug. Plasma samples, nasopharyngeal swabs, and nasal washes will be analyzed for the concentration of Conjugate A. Anti-drug antibodies (ADA) were also measured at selected time points by a validated ELISA method.

The PK parameters assessed in the PK Analysis Population using noncompartmental analysis methods in Phoenix WinNonlin (Certara) and nominal protocol specified times include: maximum plasma concentration (Cₘₐₓ), time to maximum plasma concentration (Tₘₐₓ), terminal elimination half-life (t_{1/2}), apparent clearance (CL/F), apparent volume of distribution (Vz/F), area under the plasma concentration-time curve from time 0 to time of last quantifiable sample (AUC₀₋ₜ), and area under the plasma concentration-time curve from time 0 extrapolated to infinity (AUC_{0-∞})

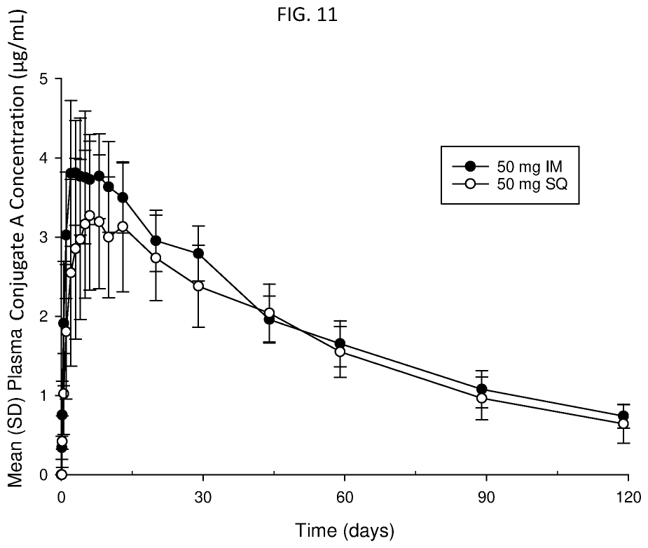

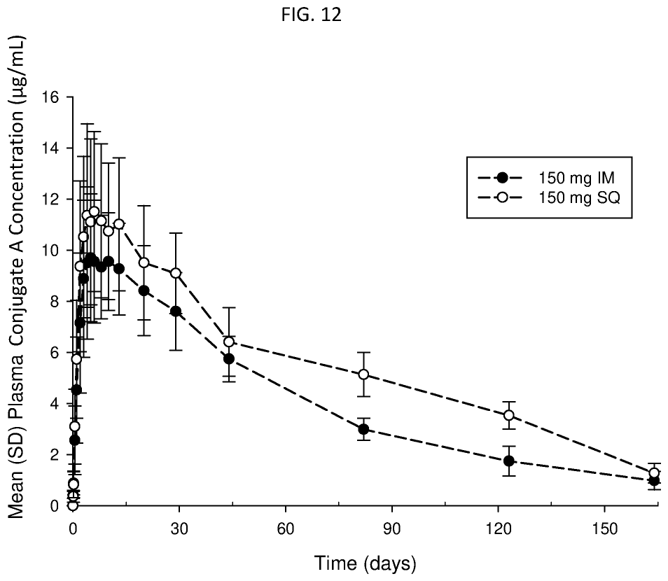

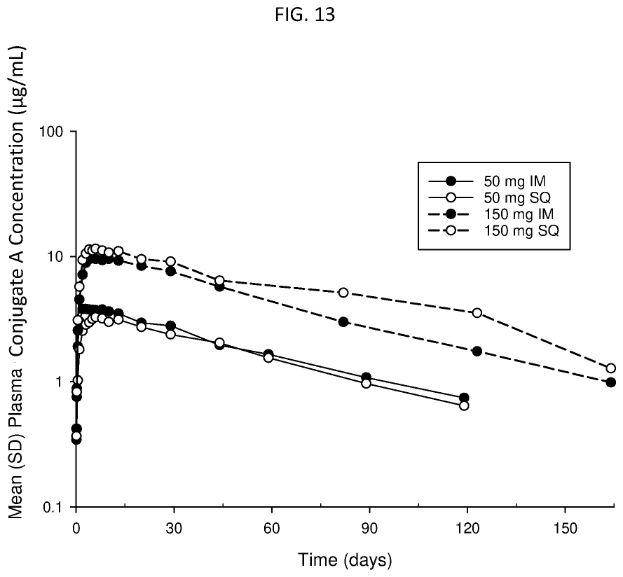

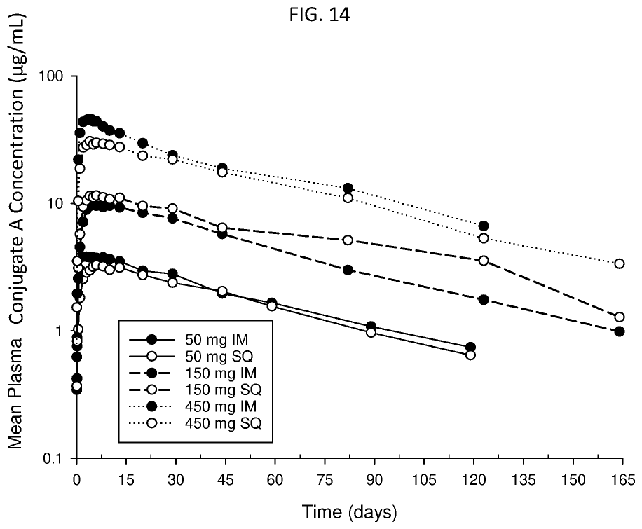

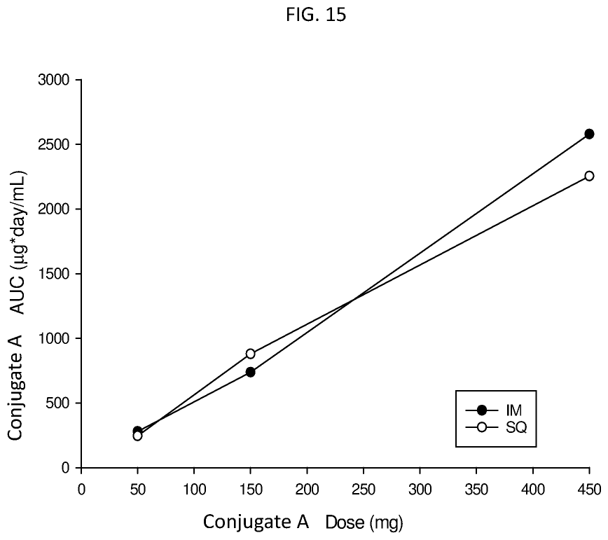

The PK parameters of each cohort are summarized in Tables 8A-8G. FIGS. 11-14 show the mean plasma concentrations of Conjugate A over time when Conjugate A is administered intramuscularly versus subcutaneously. FIG. 15 compares the mean AUC of Conjugate A when it is administered intramuscularly versus subcutaneously.

TABLE 8A
PK parameters of Cohort 1A (Treatment 1, dosage = 50 mg, IM administration).

| | | | | | | Terminal | | |
|---|---|---|---|---|---|---|---|---|
| | Tₘₐₓ | Cₘₐₓ | AUC₀₋ₜ | AUC_{0-∞} | AUC_% Extrap_obs | half-life | Tₗₐₛₜ | C4320 |
| Subject | (hr) | (μg/mL) | (hr*μg/mL) | (hr*μg/mL) | (%) | (hr) | (hr) | (μg/mL) |
| 710 | 72 | 3.75 | 5380 | 7230 | 25.5 | 1480 | 2860 | 0.437 |
| 713 | 48 | 4.39 | 5000 | 5870 | 14.8 | 998 | 2860 | 0.206 |
| 714 | 120 | 5.34 | 6410 | 8030 | 20.2 | 1240 | 2860 | 0.394 |
| 715 | 120 | 3.99 | 5350 | 6880 | 22.2 | 1350 | 2860 | 0.366 |
| 716 | 48 | 3.84 | 4200 | 4760 | 11.6 | 848 | 2860 | 0.115 |
| 717 | 192 | 3.92 | 5700 | 7390 | 22.8 | 1390 | 2860 | 0.41 |
| 718 | 240 | 2.91 | 4680 | 6070 | 22.9 | 1360 | 2860 | 0.338 |
| 719 | 48 | 4.9 | 6240 | 7420 | 15.9 | 1050 | 2860 | 0.284 |
| N | 8 | 8 | 8 | 8 | 8 | 8 | 8 | 8 |
| Mean | 111 | 4.13 | 5370 | 6710 | 19.5 | 1210 | 2860 | 0.319 |
| SD | 72.5 | 0.748 | 748 | 1060 | 4.84 | 223 | 0 | 0.111 |
| CV % | 65.3 | 18.1 | 13.9 | 15.9 | 24.9 | 18.4 | 0 | 34.8 |
| Min | 48 | 2.91 | 4200 | 4760 | 11.6 | 848 | 2860 | 0.115 |
| Median | 96 | 3.96 | 5370 | 7050 | 21.2 | 1300 | 2860 | 0.352 |
| Max | 240 | 5.34 | 6410 | 8030 | 25.5 | 1480 | 2860 | 0.437 |
| Geometric | 92.3 | 4.07 | 5330 | 6620 | 18.9 | 1200 | 2860 | 0.296 |
| Mean | | | | | | | | |
| Geometric | 72.1 | 18.6 | 14.2 | 17.1 | 27.8 | 19.8 | 0 | 47.6 |
| CV % | | | | | | | | |

${a}^{} AUC\_ \% Extrap\_obs = ( \frac{{AUC}_{0 - ∞} - {AUC}_{0 - t}}{{AUC}_{0 - ∞}} ) \times 100 \%$ ^{b} Tₗₐₛₜ = Time of the last quantifiable timepoint
^{c} C4320 = Concentration of Conjugate A predicted at day 180

TABLE 8B
PK parameters of Cohort 1B (Treatment 1, dosage = 50 mg, SQ administration).

| | | | | | AUC_% | Terminal | | |
|---|---|---|---|---|---|---|---|---|
| | Tₘₐₓ | Cₘₐₓ | AUC₀₋ₜ | AUC_{0-∞} | Extrap_obs | half-life | Tₗₐₛₜ | C4320 |
| Subject | (hr) | (μg/mL) | (hr*μg/mL) | (hr*μg/mL) | (%) | (hr) | (hr) | (μg/mL) |
| 810 | 144 | 2.17 | 3260 | 4010 | 18.6 | 1170 | 2860 | 0.186 |
| 812 | 72 | 4.83 | 5080 | 5810 | 12.5 | 990 | 2860 | 0.18 |
| 814 | 312 | 4.46 | 6460 | 8670 | 25.5 | 1380 | 2860 | 0.5 |
| 815 | 312 | 3.81 | 5470 | 6690 | 18.2 | 1120 | 2860 | 0.297 |
| 816 | 312 | 3.14 | 4800 | 6080 | 21.1 | 1240 | 2860 | 0.32 |
| 817 | 144 | 3.63 | 5370 | 6690 | 19.8 | 1200 | 2860 | 0.329 |
| 818 | 312 | 2.4 | 3970 | 4340 | 8.65 | 711 | 2860 | 0.0886 |
| 819 | 96 | 4.08 | 4200 | 4910 | 14.6 | 1030 | 2860 | 0.177 |
| N | 8 | 8 | 8 | 8 | 8 | 8 | 8 | 8 |
| Mean | 213 | 3.57 | 4830 | 5900 | 17.4 | 1110 | 2860 | 0.26 |
| SD | 108 | 0.94 | 1000 | 1510 | 5.29 | 201 | 0 | 0.128 |
| CV % | 50.9 | 26.4 | 20.8 | 25.6 | 30.5 | 18.1 | 0 | 49.4 |
| Min | 72 | 2.17 | 3260 | 4010 | 8.65 | 71 | 2860 | 0.0886 |
| Median | 228 | 3.72 | 4940 | 5940 | 18.4 | 1150 | 2860 | 0.241 |
| Max | 312 | 4.83 | 6460 | 8670 | 25.5 | 1380 | 2860 | 0.5 |
| Geometric | 185 | 3.45 | 4730 | 5740 | 16.6 | 1090 | 2860 | 0.231 |
| Mean | | | | | | | | |
| Geometric | 66.1 | 29.2 | 21.7 | 25.8 | 35.2 | 20.3 | 0 | 57.3 |
| CV % | | | | | | | | |

TABLE 8C
PK parameters of Cohort 2A (Treatment 1, dosage = 150 mg, IM administration).

| | | | | | AUC_% | Terminal | | |
|---|---|---|---|---|---|---|---|---|
| | Tₘₐₓ | Cₘₐₓ | AUC₀₋ₜ | AUC_{0-∞} | Extrap_obs | half-life | Tₗₐₛₜ | C4320 |
| Subject | (hr) | (μg/mL) | (hr*μg/mL) | (hr*μg/mL) | (%) | (hr) | (hr) | (μg/mL) |
| 611 | 312 | 8.46 | 7260 | 19700 | 63.1 | 1550 | 1060 | 1.34 |
| 613 | 144 | 8.75 | 15300 | 17300 | 11.7 | 1220 | 3940 | 0.9 |
| 614 | 72 | 10.9 | 13900 | 14800 | 6.49 | 952 | 3940 | 0.472 |
| 615 | 96 | 13.1 | 19000 | 21100 | 10 | 1150 | 3940 | 0.978 |
| 616 | 192 | 9.83 | 14400 | 14800 | 2.52 | 650 | 3940 | 0.266 |
| 617 | 120 | 11.8 | 17400 | 19700 | 11.9 | 1180 | 3940 | 0.945 |
| 618 | 96 | 13.6 | 19300 | 21200 | 8.91 | 1090 | 3940 | 0.891 |
| 619 | 240 | 6.52 | 11500 | 13000 | 11.7 | 1320 | 3940 | 0.662 |
| N | 8 | 8 | 8 | 8 | 8 | 8 | 8 | 8 |
| Mean | 159 | 10.4 | 14800 | 17700 | 15.8 | 1140 | 3580 | 0.807 |
| SD | 83.1 | 2.43 | 4030 | 3180 | 19.4 | 265 | 1020 | 0.333 |
| CV % | 52.2 | 23.4 | 27.3 | 18 | 123 | 23.2 | 28.5 | 41.3 |
| Min | 72 | 6.52 | 7260 | 13000 | 2.52 | 650 | 1060 | 0.266 |
| Median | 132 | 10.4 | 14900 | 18500 | 10.9 | 1170 | 3940 | 0.896 |
| Max | 312 | 13.6 | 19300 | 21200 | 63.1 | 1550 | 3940 | 1.34 |
| Geometric | 142 | 10.1 | 14200 | 17400 | 10.5 | 1110 | 3340 | 0.733 |
| Mean | | | | | | | | |
| Geometric | 53.8 | 25.1 | 33 | 18.8 | 110 | 26.3 | 49.1 | 54.6 |
| CV % | | | | | | | | |

TABLE 8D
PK parameters of Cohort 2B (Treatment 1, dosage = 150 mg, SQ administration).

| | | | | | AUC_% | Terminal | | |
|---|---|---|---|---|---|---|---|---|
| | Tₘₐₓ | Cₘₐₓ | AUC₀₋ₜ | AUC_{0-∞} | Extrap_obs | half-life | Tₗₐₛₜ | C4320 |
| Subject | (hr) | (μg/mL) | (hr*μg/mL) | (hr*μg/mL) | (%) | (hr) | (hr) | (μg/mL) |
| 910 | 96 | 14.1 | 20100 | 24200 | 16.9 | 1570 | 3940 | 1.47 |
| 913 | 144 | 6.84 | 13800 | 16600 | 16.9 | 1580 | 3940 | 1.07 |
| 914 | 144 | 9.18 | 14900 | 16600 | 10.1 | 1160 | 3940 | 0.772 |
| 915 | 96 | 15.2 | 22100 | 24700 | 10.5 | 1250 | 3940 | 1.15 |
| 916 | 48 | 10.9 | 17900 | 18600 | 3.44 | 810 | 3940 | 0.448 |
| 917 | 312 | 10.4 | 17400 | 19900 | 12.5 | 1350 | 3940 | 1.07 |
| 918 | 144 | 12.6 | 19300 | 21600 | 10.8 | 1250 | 3940 | 1.04 |
| 919 | 120 | 17 | 24300 | 27200 | 10.7 | 1250 | 3940 | 1.3 |
| N | 8 | 8 | 8 | 8 | 8 | 8 | 8 | 8 |
| Mean | 138 | 12 | 18700 | 21100 | 11.5 | 1280 | 3940 | 1.04 |
| SD | 77.8 | 3.35 | 3500 | 3940 | 4.28 | 243 | 0 | 0.315 |
| CV % | 56.4 | 27.8 | 18.7 | 18.6 | 37.3 | 19 | 0 | 30.2 |
| Min | 48 | 6.84 | 13800 | 16600 | 3.44 | 810 | 3940 | 0.448 |
| Median | 132 | 11.7 | 18600 | 20800 | 10.7 | 1250 | 3940 | 1.07 |
| Max | 312 | 17 | 24300 | 27200 | 16.9 | 1580 | 3940 | 1.47 |
| Geometric | 122 | 11.6 | 18400 | 20800 | 10.5 | 1250 | 3940 | 0.989 |
| Mean | | | | | | | | |
| Geometric | 56.7 | 30.3 | 19.2 | 18.8 | 53 | 21.1 | 0 | 38.4 |
| CV % | | | | | | | | |

TABLE 8E
PK parameters of Cohort 2B (Treatment 2, dosage = 150 mg, SQ administration).

| | | | | | AUC_% | Terminal | | |
|---|---|---|---|---|---|---|---|---|
| | Tₘₐₓ | Cₘₐₓ | AUC₀₋ₜ | AUC_{0-∞} | Extrap_obs | half-life | Tₗₐₛₜ | C4320 |
| Subject | (hr) | (μg/mL) | (hr*μg/mL) | (hr*μg/mL) | (%) | (hr) | (hr) | (μg/mL) |
| 910 | 48 | 22 | 15700 | 17400 | 9.59 | 594 | 2140 | 0.152 |
| 913 | 696 | 6.9 | 7940 | 19200 | 58.6 | 1810 | 1420 | 1.48 |
| 914 | 192 | 6.84 | 7760 | 14000 | 44.6 | 1120 | 1420 | 0.658 |
| 915 | 96 | 14.4 | 14800 | 22900 | 35.3 | 836 | 1420 | 0.593 |
| 916 | 264 | 10.2 | 9760 | 14800 | 34 | 849 | 1420 | 0.394 |
| 917 | 264 | 11.1 | 11100 | 23100 | 51.8 | 1450 | 1420 | 1.45 |
| 918 | 312 | 13.8 | 12900 | 20800 | 37.9 | 936 | 1420 | 0.633 |
| 919 | 144 | 18.4 | 16300 | 25800 | 36.6 | 894 | 1420 | 0.719 |
| N | 8 | 8 | 8 | 8 | 8 | 8 | 8 | 8 |
| Mean | 252 | 12.9 | 12000 | 19700 | 38.6 | 1060 | 1510 | 0.759 |
| SD | 201 | 5.33 | 3410 | 4170 | 14.6 | 392 | 255 | 0.469 |
| CV % | 79.7 | 41.2 | 28.3 | 21.1 | 37.8 | 36.9 | 16.9 | 61.8 |
| Min | 48 | 6.84 | 7760 | 14000 | 9.59 | 594 | 1420 | 0.152 |
| Median | 228 | 12.5 | 12000 | 20000 | 37.2 | 915 | 1420 | 0.646 |
| Max | 696 | 22 | 16300 | 25800 | 58.6 | 1810 | 2140 | 1.48 |
| Geometric | 193 | 12 | 11600 | 19300 | 34.8 | 1000 | 1490 | 0.623 |
| Mean | | | | | | | | |
| Geometric | 96.1 | 44.4 | 30.3 | 22.1 | 60.2 | 35.9 | 14.6 | 83.1 |
| CV % | | | | | | | | |

TABLE 8F
PK parameters of Cohort 3A (Treatment 1, dosage = 450 mg, IM administration).

| | | | | | AUC_% | Terminal | | |
|---|---|---|---|---|---|---|---|---|
| | Tₘₐₓ | Cₘₐₓ | AUC₀₋ₜ | AUC_{0-∞} | Extrap_obs | half-life | Tₗₐₛₜ | C4320 |
| Subject | (hr) | (μg/mL) | (hr*μg/mL) | (hr*μg/mL) | (%) | (hr) | (hr) | (μg/mL) |
| 510 | 96 | 35.2 | 37500 | 38600 | 2.85 | 763 | 3940 | 0.706 |
| 512 | 72 | 67.7 | 42500 | 67100 | 36.7 | 685 | 1060 | 0.861 |
| 514 | 48 | 51.1 | 63900 | 81400 | 21.5 | 1350 | 2950 | 4.58 |
| 515 | 144 | 35.3 | 46200 | 57500 | 19.7 | 1280 | 2950 | 3.04 |
| 516 | 144 | 45 | 58500 | 72400 | 19.1 | 1200 | 2950 | 3.54 |
| 517 | 84 | 55.2 | 59000 | 71300 | 17.3 | 1190 | 2950 | 3.24 |
| 518 | 72 | 40.3 | 47700 | 58600 | 18.6 | 1140 | 2950 | 2.67 |
| 519 | 24 | 59.1 | 45100 | 48400 | 6.69 | 765 | 2950 | 0.914 |
| N | 8 | 8 | 8 | 8 | 8 | 8 | 8 | 8 |
| Mean | 85.5 | 48.6 | 50100 | 61900 | 17.8 | 1050 | 2840 | 2.44 |
| SD | 42.3 | 11.7 | 9290 | 14000 | 10.2 | 264 | 798 | 1.45 |
| CV % | 49.4 | 24.2 | 18.6 | 22.5 | 57.1 | 25.2 | 28.1 | 59.2 |
| Min | 24 | 35.2 | 37500 | 38600 | 2.85 | 685 | 1060 | 0.706 |
| Median | 78 | 48 | 47000 | 62800 | 18.8 | 1170 | 2950 | 2.85 |
| Max | 144 | 67.7 | 63900 | 81400 | 36.7 | 1350 | 3940 | 4.58 |
| Geometric | 75 | 47.4 | 49300 | 60400 | 14.4 | 1010 | 2690 | 1.98 |
| Mean | | | | | | | | |
| Geometric | 64.4 | 24.5 | 18.7 | 24.7 | 95.3 | 27.7 | 40.7 | 86.5 |
| CV % | | | | | | | | |

TABLE 8G
PK parameters of Cohort 3B (Treatment 1, dosage = 450 mg, SQ administration).

| | | | | | AUC_% | Terminal | | |
|---|---|---|---|---|---|---|---|---|
| | Tₘₐₓ | Cₘₐₓ | AUC₀₋ₜ | AUC_{0-∞} | Extrap_obs | half-life | Tₗₐₛₜ | C4320 |
| Subject | (hr) | (μg/mL) | (hr*μg/mL) | (hr*μg/mL) | (%) | (hr) | (hr) | (μg/mL) |
| 411 | 48 | 30.7 | 35300 | 37700 | 6.45 | 730 | 2950 | 0.653 |
| 412 | 96 | 48 | 76700 | 91100 | 15.8 | 1460 | 3940 | 5.66 |
| 414 | 192 | 16.6 | 33300 | 38200 | 12.9 | 1300 | 3940 | 2.04 |
| 415 | 240 | 29.2 | 43700 | 48000 | 8.94 | 1120 | 3940 | 2.12 |
| 416 | 48 | 32.5 | 46900 | 49700 | 5.72 | 855 | 3940 | 1.72 |
| 417 | 144 | 38 | 57400 | 64300 | 10.8 | 1290 | 3940 | 3.05 |
| 418 | 96 | 27 | 49600 | 53700 | 7.64 | 953 | 3940 | 2.19 |
| 419 | 96 | 39.7 | 47000 | 50300 | 6.51 | 957 | 3940 | 1.64 |
| N | 8 | 8 | 8 | 8 | 8 | 8 | 8 | 8 |
| Mean | 120 | 32.7 | 48700 | 54100 | 9.34 | 1080 | 3810 | 2.38 |
| SD | 67.9 | 9.39 | 13700 | 17200 | 3.56 | 251 | 348 | 1.48 |
| CV % | 56.6 | 28.7 | 28 | 31.7 | 38.1 | 23.2 | 9.12 | 62.2 |
| Min | 48 | 16.6 | 33300 | 37700 | 5.72 | 730 | 2950 | 0.653 |
| Median | 96 | 31.6 | 47000 | 50000 | 8.29 | 1040 | 3940 | 2.08 |
| Max | 240 | 48 | 76700 | 91100 | 15.8 | 1460 | 3940 | 5.66 |
| Geometric | 104 | 31.4 | 47200 | 52100 | 8.8 | 1060 | 3800 | 2.04 |
| Mean | | | | | | | | |
| Geometric | 63.9 | 32.6 | 26.9 | 29 | 37.5 | 24 | 10.2 | 66.9 |
| CV % | | | | | | | | |

### Example 3: Population Pharmacokinetic Model of Conjugate A

A Phase 1 single ascending dose (SAD) study is being conducted in healthy volunteers evaluating safety, tolerability, and pharmacokinetics of four dose levels of Conjugate A administered subcutaneously (SQ) or intramuscularly (IM). The Conjugate A used in this study has a DAR of 4.5. Preliminary results from the SAD study were analyzed through non-linear mixed effects modelling.

A total of 66 healthy volunteers dosed on placebo (N=3 per cohort) or Conjugate A (N=8 per cohort, at 50 mg, 150 mg, or 450 mg of Conjugate A administered SQ or IM) were included in the pharmacokinetic analysis.

Conjugate A concentrations were sampled in plasma and quantified using a hybrid LCMS method with a polyclonal antibody to Conjugate A, with low cross-reactivity to human Fc alone. Preliminary results were analysed through non-linear mixed effects modelling. The overall procedures for the development of a population pharmacokinetic model for Conjugate A included exploratory data analysis, and base structural model development. Using the software NONMEM version 7.3, the first-order conditional estimation with interaction method was applied during all stages of the model development process. The observed PK was adequately described by a linear one-compartment disposition model with first-order absorption (Table 9). The half-life of Conjugate A was 6 to 8 weeks.

TABLE 9
Parameter estimates in final population pharmacokinetic model of Conjugate A.

| | Population | | Shrinkage |
|---|---|---|---|
| Parameters | Estimate | RSE (%) | (%) |
| Kₐ (/h) | 0.02493 | 10.53 | N.A. |
| CL/F (L/h) | 0.00826 | 4.74 | N.A. |
| equivalent T_{1/2} (days) ^{a} | 45.91 | | |
| Vₒ/F (L) | 13.13 | 5.66 | N.A. |
| IIV of Kₐ (CV%) | 54 | 11.81 | 1.3 |
| IIV of CL (CV%) | 19.4 | 14.69 | 9.8 |
| IIV of V_{c} (CV%) | 27.6 | 14.05 | 0.51 |
| Correlation between IIV of CL and V_{c} | 0.6218 | 20.32 | N.A. |
| Proportional residual error (CV %) | 7.96 | 12.79 | 7.9 |
| Additive residual error (μg/mL) | 0.3109 | 20.22 | |

RSE: relative standard error; CL: clearance; V_{c}: central volume of distribution; IIV: inter-individual variability, estimates in CV % calculated as exp(variance-1)^{1/2*}100% and RSE calculated as (stderr/variance)/2*100%.
${a}^{} : {θ}_{{T}_{1 / 2}} = log ( 2 ) * \frac{{θ}_{{V}_{c}}}{{θ}_{CL}}$

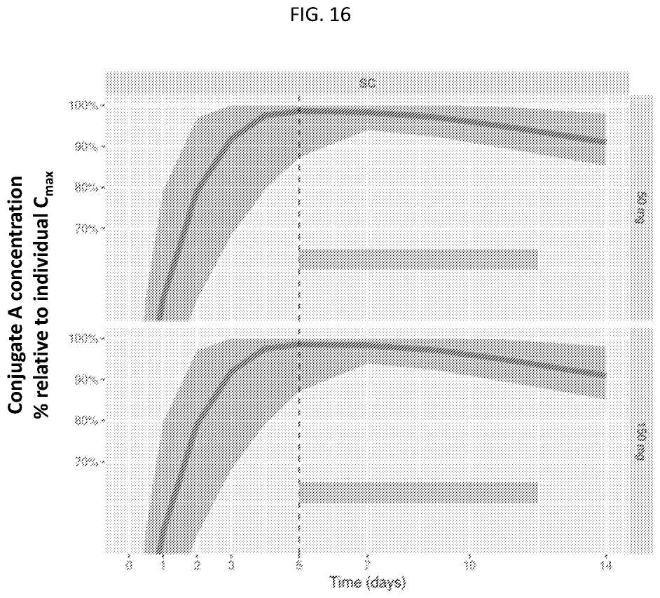

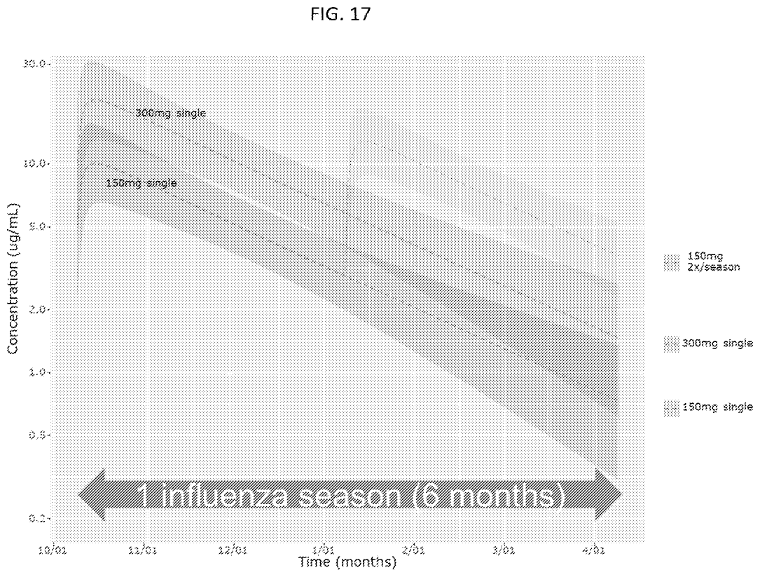

All participants were predicted to reach 90% of Cₘₐₓ at day 5 post-administration (FIG. 16). Model predicted plasma concentrations for 150 mg given once or twice on day 1 (as 300 mg) show slow concentration peak to trough decline of approximately 1 Log over flu season, whereas giving a second 150 mg dose mid flu season to subjects who received a 150 mg dose on day 1 leads to accumulation and reduced peak to trough variation (FIG. 17).

Thus, Conjugate A showed an extended half-life of 6 to 8 weeks, which could provide seasonal prevention with one or two doses per season.

### Example 4: Pharmacokinetics of Conjugate a in Humans Infected with Influenza Over Time

In this study, a single dose of 50 mg or 150 mg of Conjugate A was administered subcutaneously (SQ) to human subjects, followed by influenza virus inoculation, in order to evaluate the pharmacokinetics of Conjugate A as a function of time in human subjects infected with influenza. The Conjugate A used in this study has a DAR of 4.5.

After a single SQ dose of 50 mg and 150 mg of Conjugate A to human subjects, quantifiable Conjugate A plasma concentrations were observed at the first post-dose sampling time point (Day −5 [relative to virus inoculation], 2 hours [relative to Conjugate A dosing]) for all participants (Tables 10A and 10B). Human subjects were then challenged with inoculation of influenza virus on Day 0. Mean plasma concentrations of Conjugate A increased gradually until maximum levels were reached at Day 0 (relative to virus inoculation), 120 hours (relative to Conjugate A dosing) for both 50 mg and 150 mg. Thereafter, plasma concentrations gradually declined with fluctuations for several subjects, and additional Conjugate A plasma peaks could be observed. Notably, the single SQ dose of 50 mg of Conjugate A maintained a mean plasma concentration of 387 ng/ml after 180 days post administration of Conjugate A (Table 10B). In summary, the mean Conjugate A plasma concentrations for the 150 mg dose were higher over the whole pharmacokinetic profile than mean Conjugate A plasma concentrations for the 50 mg dose (Table 11).

TABLE 10A
Summary of Conjugate A plasma concentrations over 180 days in humans after 150 mg SQ administration.

| | Plasma concentrations of Conjugate A (ng/ml) | | | | | | | | | | | | | |
|---|---|---|---|---|---|---|---|---|---|---|---|---|---|---|
| | Day −5 | Day | Day | Day | Day | Day | Day | Day | Day | Day | Day | Day | Day | Day |
| | pre- | −5 | −5 | −4 | −2 | 0 | 1 | 2 | 3 | 17 | 28 | 60 | 120 | 180 |
| | dose | 2 h | 4 h | 24 h | 72 h | 120 h | 144 h | 168 h | 192 h | 528 h | 792 h | 1560 h | 3000 h | 4440 h |
| n | 28 | 28 | 28 | 28 | 28 | 28 | 28 | 28 | 28 | 27 | 26 | 26 | 27 | 28 |
| Mean | BQL | 478 | 1141 | 7190 | 12237 | 14050 | 13180 | 13930 | 13613 | 13244 | 10368 | 6664 | 3364 | 1407 |
| SD | — | 291 | 819 | 3500 | 3850 | 3758 | 3876 | 3548 | 3747 | 4873 | 3841 | 1813 | 996 | 484 |
| Mir | BQL | 135 | 237 | 2510 | 5950 | 7140 | 5180 | 5520 | 5050 | 6500 | 4290 | 3720 | 1310 | 297 |
| Median | BQL | 417 | 859 | 6250 | 11800 | 13800 | 11650 | 13700 | 13550 | 12600 | 9315 | 6225 | 3180 | 1255 |
| Max | BQL | 1430 | 4150 | 18300 | 21400 | 21700 | 20600 | 20000 | 22700 | 29600 | 20000 | 11400 | 5450 | 2260 |
| % CV | — | 61.0 | 71.8 | 48.7 | 31.5 | 26.7 | 29.4 | 25.5 | 27.5 | 36.8 | 37.0 | 27.2 | 29.6 | 34.4 |
| Geometric | — | 414 | 929 | 6483 | 11666 | 13553 | 12595 | 13425 | 13069 | 12471 | 9731 | 6443 | 3210 | 1311 |
| Mean | | | | | | | | | | | | | | |

Days relative to challenge influenza virus inoculation (Day 0), hours relative to Conjugate A dosing (Day −5).
BQL: Below Quantification Limit (<50 ng/ml).

TABLE 10B
Summary of Conjugate A plasma concentrations over 180 days in humans after 50 mg SQ administration.

| | Plasma concentrations of Conjugate A (ng/ml) | | | | | | | | | | | | | |
|---|---|---|---|---|---|---|---|---|---|---|---|---|---|---|
| | Day -5 | Day | Day | Day | Day | Day | Day | Day | Day | Day | Day | Day | Day | Day |
| | pre- | −5 | −5 | −4 | −2 | 0 | 1 | 2 | 3 | 17 | 28 | 60 | 120 | 180 |
| | dose | 2 h | 4 h | 24 h | 72 h | 120 h | 144 h | 168 h | 192 h | 528 h | 792 h | 1560 h | 3000 h | 4440 h |
| n | 2 | 2 | 2 | 2 | 2 | 2 | 2 | 2 | 2 | 0 | 1 | 2 | 2 | 2 |
| Mean | BQL | 315 | 705 | 2570 | 4175 | 5245 | 3870 | 4975 | 5125 | — | — | 1840 | 590 | 387 |
| SD | 0 | 254 | 658 | 1527 | 2312 | 2737 | 665 | 1817 | 2256 | — | — | 509 | 257 | 98.3 |
| Min | — | — | — | — | — | — | — | — | — | — | — | — | — | — |
| Median | — | — | — | — | — | — | — | — | — | — | — | — | — | — |
| Max | — | — | — | — | — | — | — | — | — | — | — | — | — | — |
| % CV | — | — | — | — | — | — | — | — | — | — | — | — | — | — |
| Geometric | — | — | — | — | — | — | — | — | — | — | — | — | — | — |
| Mean | | | | | | | | | | | | | | |

Days relative to challenge virus inoculation (Day 0), hours relative to Conjugate A dosing (Day −5).
BQL: Below Quantification Limit (<50 ng/ml).

TABLE 11
Summary of Conjugate A pharmacokinetics in human subjects
after a single SQ dose of 150 mg or 50 mg of Conjugate A.

| PK parameters | Single SQ | Single SQ |
|---|---|---|
| (mean [SD], Tₘₐₓ: | dose of 150 mg | dose of 50 mg |
| median [range]) | Conjugate A | Conjugate A |
| n | 27^{b} | 2 |
| Cₘₐₓ, ng/mL | 16153 [4245] | 5435 [2468] |
| Tₘₐₓ, day^{a} | 6.79 | 4.96, 6.89^{c} |
| | [2.99-21.94] | |
| AUC₀₋ₜ, | 1060136 [289137] | 297238 [87999] |
| ng · day/mL | | |
| AUC_{120-192 h}, | 40982 [10555] | 13998 [4892] |
| ng · day/mL | | |
| AUC_{0-∞}, | 1151498 [317687] | 323895 [94789] |
| ng · day/mL | | |
| λ_{z}, 1/day | 0.0138 [0.00248] | 0.0145 [<0.00001] |
| t_{1/2}, day | 51.4 [6.9] | 47.8 [0.0] |

Note:
time related parameters are relative to Conjugate A dosing.
^{a}Only n, min, median, and max are reported for Tₘₐₓ.
^{b}n = 26 for AUC_{0-∞} and t_{1/2}.
^{c}Individual values are shown for Tₘₐₓ where n = 2.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. All publications, patents, and patent applications mentioned in the above specification are hereby incorporated by reference to the same extent as if each individual publication, patent or patent application was specifically and individually indicated to be incorporated by reference in its entirety.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

1. A method for inhibiting, reducing, or shortening influenza viral replication or infection in a human subject, the method comprising or consisting of subcutaneously or intramuscularly administering a dose of a conjugate of Formula (I) to the human subject in an amount of 50 mg to 900 mg, wherein the conjugate of Formula (I) has the following structure: 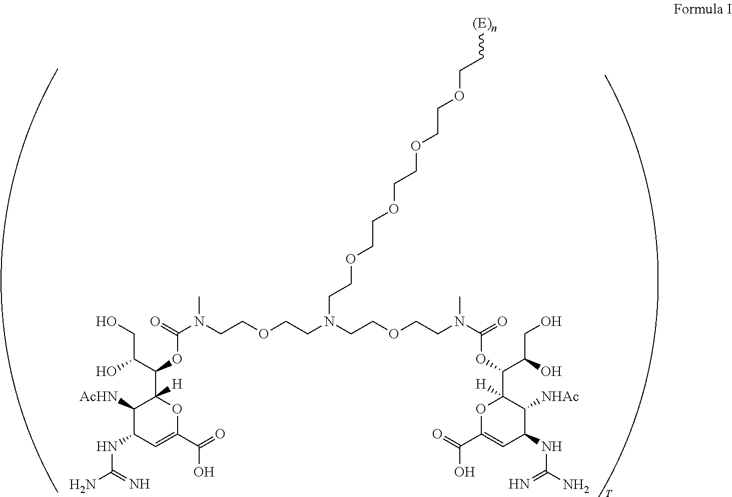
wherein each E is an Fc domain monomer;
n is 2;
T is an integer from 3 to 6; and
the squiggly line indicates a covalent attachment to a nitrogen atom of a solvent-exposed lysine or to a sulfur atom of a solvent-exposed cysteine of an E.

2. A method for treating a human subject, the method comprising or consisting of subcutaneously or intramuscularly administering a dose of a conjugate of Formula (I) in an amount of 50 mg to 900 mg, wherein the conjugate of Formula (I) has the following structure.

3. The method of claim 2, wherein the human subject has an influenza infection.

4. A method for reducing time to symptom resolution in a human subject, reducing the influenza viral load area under the curve (VL-AUC) in a human subject having an influenza infection, or reducing the peak influenza viral load time in a human subject having an influenza infection, the method comprising or consisting of subcutaneously or intramuscularly administering a dose of a conjugate of Formula (I) in an amount of 50 mg to 900 mg.

5. A method for reducing the time to a confirmed negative influenza test in a human subject having an influenza infection, the method comprising or consisting of subcutaneously or intramuscularly administering a dose of a conjugate of Formula (I) in an amount of 50 mg to 900 mg.

6. A method for reducing total clinical symptoms score (TSS-AUC) in a human subject having an influenza infection, the method comprising or consisting of subcutaneously or intramuscularly administering a dose of a conjugate of Formula (I) in an amount of 50 mg to 900 mg.

7. A method for reducing peak of TSS in a human subject having an influenza infection, the method comprising or consisting of subcutaneously or intramuscularly administering a dose of a conjugate of Formula (I) in an amount of 50 mg to 900 mg.

8. A method for inhibiting, reducing, or shortening influenza viral replication or infection in a human subject, the method comprising or consisting of:
(a) subcutaneously or intramuscularly administering a first dose of a conjugate of Formula (I) to the human subject in an amount of 50 mg to 900 mg; and
(b) subcutaneously or intramuscularly administering a second dose of a conjugate of Formula (I) to the human subject in an amount of 50 mg to 900 mg;
wherein the first and second doses are administered from 60 to 120 days apart.

9. A method for treating a human subject, the method comprising or consisting of:
(a) subcutaneously or intramuscularly administering a first dose of a conjugate of Formula (I) in an amount of 50 mg to 900 mg; and
(b) subcutaneously or intramuscularly administering a second dose of a conjugate of Formula (I) in an amount of 50 mg to 900 mg;
wherein the first and second doses are administered from 60 to 120 days apart.

10. The method of claim 9, wherein the human subject has an influenza infection.

11. A method for reducing time to symptom resolution in a human subject, reducing the influenza viral load area under the curve (VL-AUC) in a human subject having an influenza infection, or reducing the peak influenza viral load time in a human subject having an influenza infection, the method comprising or consisting of:
(a) subcutaneously or intramuscularly administering a first dose of a conjugate of Formula (I) in an amount of 50 mg to 900 mg; and
(b) subcutaneously or intramuscularly administering a second dose of a conjugate of Formula (I) in an amount of 50 mg to 900 mg;
wherein the first and second doses are administered from 60 to 120 days apart.

12. A method for reducing the time to a confirmed negative influenza test in a human subject having an influenza infection, the method comprising or consisting of:
(a) subcutaneously or intramuscularly administering a first dose of a conjugate of Formula (I) in an amount of 50 mg to 900 mg; and
(b) subcutaneously or intramuscularly administering a second dose of a conjugate of Formula (I) in an amount of 50 mg to 900 mg;
wherein the first and second doses are administered from 60 to 120 days apart.

13. A method for reducing total clinical symptoms score (TSS-AUC) in a human subject having an influenza infection, the method comprising or consisting of:
(a) subcutaneously or intramuscularly administering a first dose of a conjugate of Formula (I) in an amount of 50 mg to 900 mg; and
(b) subcutaneously or intramuscularly administering a second dose of a conjugate of Formula (I) in an amount of 50 mg to 900 mg;
wherein the first and second doses are administered from 60 to 120 days apart.

14. A method for reducing peak of TSS in a human subject having an influenza infection, the method comprising or consisting of:
(a) subcutaneously or intramuscularly administering a first dose of a conjugate of Formula (I) in an amount of 50 mg to 900 mg; and
(b) subcutaneously or intramuscularly administering a second dose of a conjugate of Formula (I) in an amount of 50 mg to 900 mg;
wherein the first and second doses are administered from 60 to 120 days apart.

15. A method for inhibiting, reducing, or shortening influenza viral replication or infection in a human subject, the method comprising or consisting of maintaining a minimum plasma concentration of a conjugate of Formula (I) of at least 300 ng/ml over four to six months.

16. The method of claim 15, wherein the minimum plasma concentration of at least 300 ng/ml of the conjugate is maintained over six months in the subject.

17. The method of claim 15, wherein the minimum plasma concentration of at least 300 ng/ml of the conjugate is maintained over four months in the subject.

18. The method of any one of claims 1-17, wherein the conjugate is Conjugate A, wherein E has the sequence of SEQ ID NO: 76.

19. The method of any one of claims 1-18, wherein each dose comprises 150 mg of the conjugate.

20. The method of any one of claims 1-18, wherein each dose comprises 300 mg of the conjugate.

21. The method of any one of claims 1-18, wherein each dose comprises 450 mg of the conjugate.

22. The method of any one of claims 1-18, wherein each dose comprises 900 mg of the conjugate.

23. A pharmaceutical composition in unit dosage form, wherein the pharmaceutical composition comprises a conjugate of Formula (I) in an amount of 50 mg to 900 mg.

24. The pharmaceutical composition of claim 23, comprising 50 mg of the conjugate.

25. The pharmaceutical composition of claim 23, comprising 150 mg of the conjugate.

26. The pharmaceutical composition of claim 23, comprising 300 mg of the conjugate.

27. The pharmaceutical composition of claim 23, comprising 450 mg of the conjugate.

28. The pharmaceutical composition of claim 23, comprising 900 mg of the conjugate.

29. The pharmaceutical composition of any one of claims 23-28, wherein the conjugate is Conjugate A, wherein E has the sequence of SEQ ID NO: 76.

30. A kit comprising the pharmaceutical composition of any one of claims 23-29 and instructions for use in a method of any one of claims 1-17.

31. The method of any one of claims 1-17, the pharmaceutical composition of any one of claims 23-29, or the kit of claim 30, wherein the conjugate comprises an Fc domain comprising an amino acid sequence at least 95% identical to the sequence of any one of SEQ ID NO: 62, SEQ ID NO: 63, SEQ ID NO: 64, SEQ ID NO: 67, SEQ ID NO: 68, SEQ ID NO: 72, SEQ ID NO: 73, SEQ ID NO: 76, SEQ ID NO: 77, SEQ ID NO: 80, SEQ ID NO: 81, SEQ ID NO: 82, SEQ ID NO: 85, SEQ ID NO: 86, SEQ ID NO: 90, SEQ ID NO: 91, SEQ ID NO: 94, or SEQ ID NO: 95.

32. The method of any one of claims 1-17, the pharmaceutical composition of any one of claims 23-29, or the kit of claim 30, wherein the conjugate comprises an Fc domain comprising an amino acid sequence of any one of SEQ ID NO: 63, SEQ ID NO: 64, SEQ ID NO: 67, SEQ ID NO: 68, SEQ ID NO: 72, SEQ ID NO: 73, SEQ ID NO: 76, or SEQ ID NO: 77.


SEQUENCE LISTING (138 sequences)

SEQ ID NO: 1 - protein, 252 aa, Escherichia coli
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 232 aa, Escherichia coli
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 251 aa, Escherichia coli
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 231 aa, Escherichia coli
MVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 258 aa, Escherichia coli
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 6 - protein, 238 aa, Escherichia coli
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 7 - protein, 257 aa, Escherichia coli
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 8 - protein, 237 aa, Escherichia coli
MVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 9 - protein, 262 aa, Escherichia coli
MYRMQLLSCIALSLALVTNSMVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 10 - protein, 242 aa, Escherichia coli
MVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 11 - protein, 236 aa, Escherichia coli
MVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 258 aa, Escherichia coli
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 13 - protein, 238 aa, Escherichia coli
PRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 14 - protein, 257 aa, Escherichia coli
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 15 - protein, 237 aa, Escherichia coli
MVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 16 - protein, 252 aa, Escherichia coli
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 266 aa, Escherichia coli
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 18 - protein, 248 aa, Escherichia coli
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 19 - protein, 252 aa, Escherichia coli
MKWVTFISLLFLFSSAYSISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 234 aa, Escherichia coli
ISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 266 aa, Escherichia coli
MKWVTFISLLFLFSSAYSISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 22 - protein, 248 aa, Escherichia coli
ISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 23 - protein, 266 aa, Escherichia coli
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 24 - protein, 248 aa, Escherichia coli
ISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 25 - protein, 266 aa, Escherichia coli
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGGGGGSEQKLISEEDL

SEQ ID NO: 26 - protein, 248 aa, Escherichia coli
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGGGGGSEQKLISEEDL

SEQ ID NO: 27 - protein, 266 aa, Escherichia coli
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 28 - protein, 248 aa, Escherichia coli
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 29 - protein, 266 aa, Escherichia coli
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 30 - protein, 248 aa, Escherichia coli
ISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 31 - protein, 259 aa, Escherichia coli
MKWVTFISLLFLFSSAYSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 32 - protein, 245 aa, Escherichia coli
MKWVTFISLLFLFSSAYSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 231 aa, Escherichia coli
MVRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 250 aa, Escherichia coli
MKWVTFISLLFLFSSAYSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 245 aa, Escherichia coli
MGWSCIILFLVATATGVHSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 226 aa, Escherichia coli
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 226 aa, Escherichia coli
KTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 38 - protein, 259 aa, Escherichia coli
MKWVTFISLLFLFSSAYSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 39 - protein, 226 aa, Escherichia coli
  VARIANT 32: note = X is Met or Tyr
  VARIANT 34: note = X is Ser or Thr
  VARIANT 36: note = X is Thr or Glu
  VARIANT 136: note = X is Asp or Glu
  VARIANT 138: note = X is Leu or Met
  VARIANT 207: note = X is Met or Leu
  VARIANT 214: note = X is Asn or Ser
DKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 40 - protein, 226 aa, Escherichia coli
  VARIANT 136: note = X is Asp or Glu
  VARIANT 138: note = X is Leu or Met
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 226 aa, Escherichia coli
  VARIANT 136: note = X is Asp or Glu
  VARIANT 138: note = X is Leu or Met
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 226 aa, Escherichia coli
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 226 aa, Escherichia coli
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 226 aa, Escherichia coli
  VARIANT 136: note = X is Asp or Glu
  VARIANT 138: note = X is Leu or Met
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 45 - protein, 226 aa, Escherichia coli
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 46 - protein, 226 aa, Escherichia coli
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 47 - protein, 265 aa, Escherichia coli
  VARIANT 71: note = X is Met or Tyr
  VARIANT 73: note = X is Ser or Thr
  VARIANT 75: note = X is Thr or Glu
  VARIANT 175: note = X is Asp or Glu
  VARIANT 177: note = X is Leu or Met
  VARIANT 247: note = X is Met or Leu
  VARIANT 253: note = X is Asn or Ser
MGWSCIILFLVATATGVHSNVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 48 - protein, 266 aa, Escherichia coli
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 266 aa, Escherichia coli
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 266 aa, Escherichia coli
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 51 - protein, 266 aa, Escherichia coli
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 52 - protein, 266 aa, Escherichia coli
MGWSCIILFLVATATGVHSNVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 266 aa, Escherichia coli
MGWSCIILFLVATATGVHSNVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 267 aa, Escherichia coli
MGWSCIILFLVATATGVHSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSEQKLISEEDL

SEQ ID NO: 55 - protein, 267 aa, Escherichia coli
MGWSCIILFLVATATGVHSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSEQKLISEEDL

SEQ ID NO: 56 - protein, 267 aa, Escherichia coli
MGWSCIILFLVATATGVHSISAMVRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 57 - protein, 267 aa, Escherichia coli
MGWSCIILFLVATATGVHSISAMVRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 58 - protein, 275 aa, Escherichia coli
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSQRNPRLRLIRRHPTLRIPPI

SEQ ID NO: 59 - protein, 275 aa, Escherichia coli
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSQRNPRLRLIRRHPTLRIPPI

SEQ ID NO: 60 - protein, 247 aa, Escherichia coli
  VARIANT 52: note = X is Met or Tyr
  VARIANT 54: note = X is Ser or Thr
  VARIANT 56: note = X is Thr or Glu
  VARIANT 156: note = X is Asp or Glu
  VARIANT 158: note = X is Leu or Met
  VARIANT 228: note = X is Met or Leu
  VARIANT 234: note = X is Asn or Ser
  VARIANT 20: note = X is Cys or Ser
NVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 61 - protein, 247 aa, Escherichia coli
  VARIANT 52: note = X is Met or Tyr
  VARIANT 54: note = X is Ser or Thr
  VARIANT 56: note = X is Thr or Glu
  VARIANT 156: note = X is Asp or Glu
  VARIANT 158: note = X is Leu or Met
  VARIANT 228: note = X is Met or Leu
  VARIANT 234: note = X is Asn or Ser
NVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 62 - protein, 247 aa, Escherichia coli
  VARIANT 156: note = X is Asp or Glu
  VARIANT 158: note = X is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 247 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 247 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 247 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 66 - protein, 247 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 67 - protein, 247 aa, Escherichia coli
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 247 aa, Escherichia coli
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 246 aa, Escherichia coli
  VARIANT 52: note = X is Met or Tyr
  VARIANT 54: note = X is Ser or Thr
  VARIANT 56: note = X is is Thr or Glu
  VARIANT 156: note = X is Asp or Glu
  VARIANT 158: note = X is is Leu or Met
  VARIANT 228: note = X is Met or Leu
  VARIANT 234: note = X is Asn or Ser
  VARIANT 20: note = X is Cys or Ser
NVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 70 - protein, 246 aa, Escherichia coli
  VARIANT 52: note = X is Met or Tyr
  VARIANT 54: note = X is Ser or Thr
  VARIANT 56: note = X is Thr or Glu
  VARIANT 156: note = X is Asp or Glu
  VARIANT 158: note = X is Leu or Met
  VARIANT 228: note = X is Met or Leu
  VARIANT 234: note = X is Asn or Ser
NVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 71 - protein, 246 aa, Escherichia coli
  VARIANT 156: note = X is Asp or Glu
  VARIANT 158: note = X is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 246 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 246 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 246 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 75 - protein, 246 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 76 - protein, 246 aa, Escherichia coli
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 246 aa, Escherichia coli
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 246 aa, Escherichia coli
  VARIANT 51: note = X is Met or Tyr
  VARIANT 53: note = X is is Ser or Thr
  VARIANT 55: note = X is Thr or Glu
  VARIANT 155: note = X is Asp or Glu
  VARIANT 157: note = X is Leu or Met
  VARIANT 227: note = X is Met or Leu
  VARIANT 233: note = X is Asn or Ser
  VARIANT 19: note = X is Cys or Ser
VNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 79 - protein, 246 aa, Escherichia coli
  VARIANT 51: note = X is Met or Tyr
  VARIANT 53: note = X is Ser or Thr
  VARIANT 55: note = X is Thr or Glu
  VARIANT 155: note = X is Asp or Glu
  VARIANT 157: note = X is Leu or Met
  VARIANT 227: note = X is Met or Leu
  VARIANT 233: note = X is Asn or Ser
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 80 - protein, 246 aa, Escherichia coli
  VARIANT 155: note = X is Asp or Glu
  VARIANT 157: note = X is Leu or Met
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 246 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 246 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 246 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 84 - protein, 246 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 85 - protein, 246 aa, Escherichia coli
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 246 aa, Escherichia coli
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 245 aa, Escherichia coli
  VARIANT 51: note = X is Met or Tyr
  VARIANT 53: note = X is Ser or Thr
  VARIANT 55: note = X is Thr or Glu
  VARIANT 155: note = X is Asp or Glu
  VARIANT 157: note = X is Leu or Met
  VARIANT 227: note = X is Met or Leu
  VARIANT 233: note = X is Asn or Ser
  VARIANT 19: note = X is Cys or Ser
VNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 88 - protein, 245 aa, Escherichia coli
  VARIANT 51: note = X is Met or Tyr
  VARIANT 53: note = X is Ser or Thr
  VARIANT 55: note = X is Thr or Glu
  VARIANT 155: note = X is Asp or Glu
  VARIANT 157: note = X is Leu or Met
  VARIANT 227: note = X is Met or Leu
  VARIANT 233: note = X is Asn or Ser
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 89 - protein, 245 aa, Escherichia coli
  VARIANT 155: note = X is Asp or Glu
  VARIANT 157: note = X is Leu or Met
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 245 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 245 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 245 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 93 - protein, 245 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 94 - protein, 245 aa, Escherichia coli
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 245 aa, Escherichia coli
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 247 aa, Escherichia coli
  VARIANT 52: note = X is Met or Tyr
  VARIANT 54: note = X is Ser or Thr
  VARIANT 56: note = X is Thr or Glu
  VARIANT 97: note = X is Asn or Ala
  VARIANT 109: note = X is Leu or Asp
  VARIANT 111: note = X is Gln or His
  VARIANT 156: note = X is Asp or Glu
  VARIANT 158: note = X is Leu or Met
  VARIANT 228: note = X is Met or Leu
  VARIANT 234: note = X is Asn or Ser
  VARIANT 20: note = X is Cys or Ser
  VARIANT 1: note = X is Asn or absent
  VARIANT 247: note = X is Lys or absent
XVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 97 - protein, 247 aa, Escherichia coli
  VARIANT 97: note = X is Asn or Ala
  VARIANT 109: note = X is Leu or Asp
  VARIANT 111: note = X is Gln or His
  VARIANT 156: note = X is Asp or Glu
  VARIANT 158: note = X is Leu or Met
  VARIANT 234: note = X is Asn or Ser
  VARIANT 1: note = X is Asn or absent
  VARIANT 247: note = X is Lys or absent
XVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHXHYTQKSLSLSPGX

SEQ ID NO: 98 - protein, 247 aa, Escherichia coli
  VARIANT 97: note = X is Asn or Ala
  VARIANT 156: note = X is Asp or Glu
  VARIANT 158: note = X is Leu or Met
  VARIANT 1: note = X is Asn or absent
  VARIANT 247: note = X is Lys or absent
XVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGX

SEQ ID NO: 99 - protein, 247 aa, Escherichia coli
  VARIANT 97: note = X is Asn or Ala
  VARIANT 156: note = X is Asp or Glu
  VARIANT 158: note = X is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 100 - protein, 247 aa, Escherichia coli
  VARIANT 156: note = X is Asp or Glu
  VARIANT 158: note = X is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 101 - protein, 247 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 102 - protein, 247 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 103 - protein, 246 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 104 - protein, 246 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 105 - protein, 247 aa, Escherichia coli; identical to SEQ ID NO: 101

SEQ ID NO: 106 - protein, 247 aa, Escherichia coli; identical to SEQ ID NO: 102

SEQ ID NO: 107 - protein, 245 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 108 - protein, 245 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 109 - protein, 247 aa, Escherichia coli
  VARIANT 156: note = X is Asp or Glu
  VARIANT 158: note = X is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 110 - protein, 247 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 111 - protein, 247 aa, Escherichia coli
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 112 - protein, 246 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 113 - protein, 246 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 114 - protein, 247 aa, Escherichia coli; identical to SEQ ID NO: 110

SEQ ID NO: 115 - protein, 247 aa, Escherichia coli; identical to SEQ ID NO: 111

SEQ ID NO: 116 - protein, 245 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 117 - protein, 245 aa, Escherichia coli
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 118 - protein, 247 aa, Escherichia coli
  VARIANT 97: note = X is Asn or Ala
  VARIANT 109: note = X is Leu or Asp
  VARIANT 111: note = X is Gln or His
  VARIANT 155: note = X is Asp or Glu
  VARIANT 157: note = X is Leu or Met
  VARIANT 234: note = X is Asn or Ser
  VARIANT 1: note = X is Asn or absent
  VARIANT 247: note = X is Lys or absent
XVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHXHYTQKSLSLSPGX

SEQ ID NO: 119 - protein, 247 aa, Escherichia coli
  VARIANT 97: note = X is Asn or Ala
  VARIANT 156: note = X is Asp or Glu
  VARIANT 158: note = X is Leu or Met
  VARIANT 1: note = X is Asn or absent
  VARIANT 247: note = X is Lys or absent
XVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGX

SEQ ID NO: 120 - protein, 247 aa, Escherichia coli
  VARIANT 97: note = X is Asn or Ala
  VARIANT 156: note = X is Asp or Glu
  VARIANT 158: note = X is Leu or Met
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 121 - protein, 247 aa, Escherichia coli
  VARIANT 156: note = X is Asp or Glu
  VARIANT 158: note = X is Leu or Met
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 122 - protein, 247 aa, Escherichia coli
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 123 - protein, 247 aa, Escherichia coli
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 124 - protein, 246 aa, Escherichia coli
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 125 - protein, 246 aa, Escherichia coli
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 126 - protein, 247 aa, Escherichia coli; identical to SEQ ID NO: 122

SEQ ID NO: 127 - protein, 247 aa, Escherichia coli; identical to SEQ ID NO: 123

SEQ ID NO: 128 - protein, 245 aa, Escherichia coli
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 129 - protein, 245 aa, Escherichia coli
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 130 - protein, 247 aa, Escherichia coli
  VARIANT 156: note = X is Asp or Glu
  VARIANT 158: note = X is Leu or Met
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 131 - protein, 247 aa, Escherichia coli
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 132 - protein, 247 aa, Escherichia coli
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 133 - protein, 246 aa, Escherichia coli
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 134 - protein, 246 aa, Escherichia coli
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 135 - protein, 247 aa, Escherichia coli; identical to SEQ ID NO: 131

SEQ ID NO: 136 - protein, 247 aa, Escherichia coli; identical to SEQ ID NO: 132

SEQ ID NO: 137 - protein, 245 aa, Escherichia coli
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 138 - protein, 245 aa, Escherichia coli
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG